(19) United States
(12) Patent Application Publication
(10) Pub. No.: US 2026/0226190 A1
(43) Pub. Date: Aug. 6, 2026

(54) ENGINEERED ANTI-PROSTATE STEM CELL ANTIGEN FUSION PROTEIN AND USES THEREOF

(71) Applicant: CITY OF HOPE, Duarte, CA (US)

(72) Inventors: Kirstin A. ZETTLITZ, Duarte, CA (US); Anna M. WU, Duarte, CA (US)

(73) Assignee: CITY OF HOPE, Duarte, CA (US)

(21) Appl. No.: 19/154,999

(22) PCT Filed: Feb. 8, 2024
(86) PCT No.: PCT/US2024/015070

§ 371 Date: Aug. 8, 2025

Related U.S. Application Data
(60) Provisional application No. 63/483,935, filed on Feb. 8, 2023.
(60) Provisional application No. 63/499,448, filed on May 1, 2023.

(51) Int. Cl. C07K 16/30; A61K 45/06; A61K 51/10; A61P 35/00

(52) U.S. Cl. CPC C07K 16/3069; A61K 45/06; A61K 51/1072; A61K 51/1096; A61P 35/00; C07K 2317/21; C07K 2317/524; C07K 2317/526; C07K 2317/53; C07K 2317/56; C07K 2317/622; C07K 2317/92; C07K 2319/30

(57) ABSTRACT

Disclosed are engineered anti-prostate stem cell antigen (PSCA) fusion proteins and uses thereof to detect and treat cancers expressing PSCA.

### CROSS-REFERENCE TO RELATED APPLICATION(S)

This application claims the benefit of U.S. Provisional Patent Application No. 63/499,448, filed on May 1, 2023 and U.S. Provisional Patent Application No. 63/483,935, filed on Feb. 8, 2023; the contents of each of these provisional applications are incorporated by reference in their entirety.

### SEQUENCE LISTING

This application contains a ST.26 compliant Sequence Listing, which was submitted in XML format via Patent Center, and is hereby incorporated by reference in its entirety. The XML copy, created on Feb. 8, 2024, is named 0544358225WO00.xml and is 12,000 bytes in size.

### BACKGROUND

Human prostate stem cell antigen (PSCA) is a small glycosylphosphatidylinositol (GPI)-anchored cell surface glycoprotein that is expressed in epithelial cells of prostate, urinary bladder, kidney, stomach and placenta. PSCA is overexpressed in prostate cancer and expression levels correlate with Gleason score and poor prognosis. PSCA is also highly expressed in prostate cancer metastases to lymph nodes and bone. PSCA expression is elevated in bladder cancer and pancreatic carcinoma. Previous work described the anti-human PSCA antibody 1G8 that specifically targets PSCA-expressing cells and inhibits tumor growth in preclinical models.

The mouse IgG 1G8 was humanized by CDR-grafting (2B3) and affinity matured using yeast-display. Based on two affinity matured variants (A2 and A11) small, bivalent antibody fragment were engineered; the 80 kDa minibody (A11 Mb), a scFv-CH3 dimer and the cys-diabody (A2cDb), a 50 kDa scFv dimer functionalism with C-terminal cysteine residues. These antibody fragments display pharmacokinetics that are optimal for molecular imaging applications such as short plasma half-life, and good tumor penetration. However, renal clearance prevents the use for radioimmunotherapy because of nephrotoxicity. Full-length IgGs exhibit clearance (other than target-mediated) mainly through the hepatobiliary route and the liver is less radiosensitive. Nevertheless, the long plasma half-life of IgGs (up to three weeks in humans) causes hematological toxicities when used in radioimmunotherapy. Therefore, antibodies having improved properties suitable for diagnostic and therapeutic uses need to be developed.

### SUMMARY

In one aspect, this disclosure relates to a genetically engineered anti-prostate stem cell antigen (PSCA) scFv-Fc fusion protein. The anti-PSCA scFv-Fc fusion protein comprises two peptides which form a homodimer, and each peptide comprises variable domains VH and VL of an anti-PSCA antibody, and a truncated hinge and fragment crystallizable (Fc) region. In some embodiments, the variable domains are arranged in the order of VH-VL. In some embodiments, the variable domains VH and VL are connected by a glycine-rich linker. In some embodiments, the linker is about 10-25 amino acids such as 10 amino acids, 11 amino acids, 12 amino acids, 13 amino acids, 14 amino acids, 15 amino acids, 16 amino acids, 17 amino acids, 18 amino acids, 19 amino acids, 20 amino acids, 21 amino acids, 22 amino acids, 23 amino acids, 24 amino acids, or 25 amino acids. In some embodiments, the linker has a sequence of ((G4S) 2-GGSAQ) (SEQ ID NO: 1).

In some embodiments, the FcRn binding region of the Fc region contains one or more first point mutations. In some embodiments, the one or more first point mutations reduces hematological toxicity and plasma half life. In some embodiments, the one or more first point mutations is selected from H310A, H435Q, or both H310A and H435Q. In some embodiments, the one or more first point mutations is H310A. In some embodiments, the one or more first point mutations is H435Q. In some embodiments, the one or more first point mutations is H310A and H435Q. In some embodiments, the Fc region comprises a second point mutation that is N297Q, and wherein the second point mutation removes an N-glycosylation site. In some embodiments, the truncated hinge and Fc region is derived from human IgG2. In some embodiments, the truncated hinge and Fc region comprises a third point mutation that is K322A. In some embodiments, the third point mutation causes reduced effector functions (e.g., ADCC, CDC). In certain embodiments, the anti-PSCA scFv-Fc fusion protein comprises a first point mutation H310A, no second point mutation, and optionally, the third point mutation (K322A). In certain embodiments, the anti-PSCA scFv-Fc fusion protein comprises a first point mutation H310A, the second point mutation (N297Q), and optionally, the third point mutation (K322A). In certain embodiments, the anti-PSCA scFv-Fc fusion protein comprises two first point mutations: H310A and H435Q, no second point mutation, and optionally, the third point mutation (K322A). In certain embodiments, the anti-PSCA scFv-Fc fusion protein comprises two first point mutations: H310A and H435Q, the second point mutation (N297Q), and optionally, the third point mutation (K322A).

In some embodiments, each peptide of the scFv-Fc fusion protein has an amino acid sequence at least 80%, at least 85%, at least 90%, at least 95%, at least 98%, or at least 99% identical to SEQ ID NO: 2, SEQ ID NO: 3, or SEQ ID NO: 5. In some embodiments, the scFv-Fc fusion protein is conjugated to an effector moiety including labeling moieties such as detectable markers including radioactive labels or fluorescent labels, and a therapeutic moiety. In certain embodiments, the therapeutic moiety includes a cytotoxic agent, an anti-tumor drug, a toxin, a radioactive agent, a cytokine, a second protein, an antibody, a radionuclide, or an enzyme.

In a related aspect, this disclosure relates to a pharmaceutical composition comprising an effective amount of one or more scFv-Fc fusion proteins, or scFv-Fc fusion protein and effector moiety conjugates disclosed herein. In some embodiments, the pharmaceutical composition further comprises one or more pharmaceutically acceptable carriers, excipients, and/or stabilizers. In some embodiments, the pharmaceutical composition further comprises one or more additional therapeutic agents including, for example, chemotherapeutic agents, cytotoxic agents, cytokines, growth inhibitory agents, radionuclides, and anti-hormonal agents. The therapeutic agent can be conjugated to the scFv-Fc fusion protein. In some embodiments, the pharmaceutical composition is formulated suitable for intravenous, intramuscular, intraperitoneal, intracerobrospinal, subcutaneous, intraarticular, intrasynovial, intrathecal, oral, topical, or inhalation administration.

In another aspect, disclosed is a method of treating or preventing a cancer expressing PSCA comprising administering to a subject an effective amount of one or more scFv-Fc fusion proteins, scFv-Fc fusion protein and effector moiety conjugates, or pharmaceutical compositions disclosed herein. In some embodiments, the method further entails administering to the subject one or more additional therapeutic agents including, for example, chemotherapeutic agents, cytotoxic agents, cytokines, growth inhibitory agents, radionuclides, and anti-hormonal agents. In some embodiments, the cancer expressing PSCA includes prostate cancer, pancreatic cancer, and bladder cancer.

In another aspect, disclosed herein is a method of detecting cancer expressing PSCA in a subject. The method entails administering one or more scFv-Fc fusion proteins disclosed herein to a subject, measuring the level of the scFv-Fc fusion proteins in the subject, comparing the level of the scFv-Fc fusion proteins with that of a healthy subject or with an average level of a healthy population, wherein an elevated level of the scFv-Fc fusion proteins in the subject indicating the presence of cancer. In some embodiments, the scFv-Fc fusion protein is conjugated with a labeling moiety. In some embodiments, the labeling moiety comprises one or more radioactive isotopes such as ³²P, ^{99m}Tc, ¹¹¹In, ¹⁸F, ⁶⁴Cu, and ⁸⁹Zr, fluorescent dyes, electron-dense reagents, enzymes, biotin, digoxigenin, or haptens and proteins which can be made detectable. In some embodiments, the cancer expressing PSCA includes prostate cancer, pancreatic cancer, and bladder cancer.

In another aspect, disclosed herein is a method of determining the prognosis of treating a cancer expressing PSCA in a subject. The method entails administering one or more scFv-Fc fusion proteins disclosed herein to a subject, measuring the level of the scFv-Fc fusion proteins in the subject, comparing the level of the scFv-Fc fusion proteins before and after the subject receives a cancer therapy, wherein a decreased level of the scFv-Fc fusion proteins in the subject after receiving the cancer therapy indicating that the cancer therapy is effective. In some embodiments, the scFv-Fc fusion protein is conjugated with a labeling moiety. In some embodiments, the labeling moiety comprises one or more radioactive isotopes such as ³²P, ^{99m}Tc, ¹¹¹In, ¹⁸F, ⁶⁴Cu, and ⁸⁹Zr, fluorescent dyes, electron-dense reagents, enzymes, biotin, digoxigenin, or haptens and proteins which can be made detectable. In some embodiments, the cancer expressing PSCA includes prostate cancer, pancreatic cancer, and bladder cancer.

In another aspect, disclosed herein is a method of imaging a cancer expressing PSCA in a subject. The method entails administering one or more scFv-Fc fusion proteins disclosed herein to a subject and imaging the subject to determine the location and size of the tumor. In some embodiments, the scFv-Fc fusion protein is conjugated with a labeling moiety. In some embodiments, the labeling moiety comprises one or more radioactive isotopes such as ³²P, ^{99m}Tc, ¹¹¹In, ¹⁸F, ⁶⁴Cu, and ⁸⁹Zr, fluorescent dyes, electron-dense reagents, enzymes, biotin, digoxigenin, or haptens and proteins which can be made detectable. In some embodiments, the cancer expressing PSCA includes prostate cancer, pancreatic cancer, and bladder cancer.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a schematic of novel anti-PSCA A2scFv-Fc2 variants illustrating the different PK-modulated scFv-Fc2 variants (DM=double mutant, aG=aglycosylated, SM=single mutant).

FIG. 2 shows an SDS-PAGE analysis of purified A2 scFv-Fc2 variants under non-reducing and reducing conditions, wherein the NuPAGE 4-12% Bis-Tris gel was stained with Coomassie Blue: the same molecular weight was observed for the scFv-Fc2 fragments (110 kDa) except for the aglycosylated A2aG (100 kDa).

FIGS. 3A-3B show size exclusion chromatography of purified A2 scFv-Fc2 SM (FIG. 3A) and A2 scFv-Fc2 DMaG (FIG. 3B).

FIG. 4 shows ELISA data for A2SM and A2DMaG antigen-specific binding. Saturation binding curves of 1 of 3 independent experiments is shown, duplicates were fitted using the one site-specific binding model (GraphPad Prism 8).

FIGS. 5A-5B show antigen-specific cell binding data of A2SM and A2DMaG. Both A2SM (FIG. 5A) and A2DMaG (FIG. 5B) show specific and dose-dependent binding to RM9-hPSCA cells and no binding to RM9 cells. Bound antibody was detected with goat anti-humanIgG-AF647.

FIGS. 6A-6C show saturation binding data on PSCA-expressing cells (RM9-hPSCA). Serial dilutions of A2SM (FIG. 6A) and A2DMaG (FIG. 6B), respectively, were incubated with RM9-hPSCA cells until equilibrium and analyzed by flow cytometry, the resulting saturation binding curve (FIG. 6C) was fitted (One-site specific binding, GraphPad Prism) to calculate the K_{d}.

FIGS. 7A-7B show DFO-conjugation data of A2sM and A2DMaG. SDS-PAGE analysis of DFO-conjugated protein shows a slight shift to higher molecular weight for the DFO-A2SM and DFO-A2DMaG (FIG. 7A). The SEC analysis confirms that the integrity of the proteins was not impacted by conjugation of the DFO chelator (FIG. 7B).

FIGS. 8A-8C show ⁸⁹Zr-A2SM immunoPET data in a syngeneic prostate cancer model in hPSCA KI mice. FIG. 8A shows a schematic of A2scFvFc2SM conjugation with p-SON-DFO followed by chelation of ⁸⁹Zr. ⁸⁹Zr-A2SM (45 μCi/10 μg) was injected i.v. into hPSCA kl mice bearing PSCA-negative RM9 (FIG. 8B) or PSCA-positive RM9-hPSCA (FIG. 8C) tumors s.Fc. and serial PET/CT scans acquired at indicated time points. Representative scans are shown as whole-body maximum-intensity projection PET/CT overlay.

FIG. 9 shows an annotated nucleic acid sequence and amino acid sequence of A2scFvFc2SM (SEQ ID NOs 3 and 4).

FIG. 10 shows an annotated nucleic acid sequence and amino acid sequence of A2scFvFc2DMaG (SEQ ID NOs. 5 and 6).

FIGS. 11A-11C show characterization of the conjugation of A2DM. FIG. 11A shows a schematic of random and site-specific conjugation of A2DM with IRDye800CW. FIG. 11B shows SDS-PAGE analysis of unconjugated A2DM compared to A2DM-NHS800 and A2DM-mal800 (both DPR 0.5). Gels were stained with coomassie (left panel) or visualized using a NIR imaging system (Odyssey DLx, right panel). FIG. 11C shows size exclusion chromatography shows IRDye800-conjugated proteins eluting as a single peak (retained dimeric conformation) and the fluorescent dye co-eluting, confirming successful conjugation.

FIGS. 12A-12D shows antigen binding affinity and specificity. FIGS. 12A and 12B show kinetics of binding to immobilized hPSCA-mFc by ELISA. Binding to antigen-positive RM9-hPSCA shows A2DM-NHS800 impacted by higher DPR while A2DM-mal800 binds similarly to unconjugated A2DM (FIGS. 12C and 12D).

FIG. 13 shows ⁸⁹Zr-immunoPET imaging of RM9/RM9-hPSCA tumors in hPSCA KI mice. FIG. 13A shows representative immunoPET/CT overlays at 4, 24, and 48 h p.i. of 10 μg/50 μCi of ⁸⁹Zr-A2DM, ⁸⁹Zr-A2DM-NHS800, or ⁸⁹Zr-A2DM-mal800. FIG. 13B shows ex vivo biodistribution at 48 h post injection (p.i.).

FIG. 14 shows near-infrared fluorescent imaging of hPSCA KI mice bearing RM9-hPSCA tumors. Mice were imaged post-mortem with the skin removed to expose the antigen-negative RM9 (left shoulder) and antigen-positive RM9-hPSCA (right shoulder) tumors (dorsal images, left panel). Ventral images (right panels) show clearance of the tracer through the liver.

### DETAILED DESCRIPTION

Prostate Stem Cell antigen (PSCA) and Cancer

Prostate stem cell antigen (PSCA) is a small glycosylphosphatidylinositol (GPI)-anchored cell surface glycoprotein that is expressed in epithelial cells of prostate, urinary bladder, kidney, stomach and placenta. PSCA is over-expressed in prostate cancer and expression levels correlate with Gleason score and poor prognosis. PSCA is also highly expressed in prostate cancer metastases to lymph nodes and bone. PSCA expression is elevated in bladder cancer and pancreatic carcinoma. Since PSCA is overexpressed in cancers of the prostate and bladder with minimal expression in normal tissues, it is a promising target for antibody-based therapies such as radioimmunotherapy (RIT). Theranostic antibody fragments that specifically bind PSCA can be used for the delivery of diagnostic or therapeutic payloads to cancers that express PSCA, such as prostate cancer, pancreatic cancer and bladder cancer.

The anti-human PSCA antibody 1G8 has been shown to specifically target PSCA-expressing cells and inhibit tumor growth in preclinical models. The mouse IgG 1G8 was humanized by CDR-grafting (2B3) and affinity matured using yeast-display. Based on two affinity matured variants (A2 and A11), small, bivalent antibody fragments were engineered; the 80 kDa minibody (A11 Mb), a scFv-CH3 dimer, and the cys-diabody (A2cDb), a 50 kDa scFv dimer functionalized with C-terminal cysteine residues. These antibody fragments were observed to display pharmacokinetics that are optimal for molecular imaging application (short plasma half-life, good tumor penetration). However, renal clearance prevents the use of these antibody fragments for radioimmunotherapy because of nephrotoxicity. Full-length IgGs exhibit clearance (other than target-mediated) mainly through the hepatobiliary route and the liver is less radiosensitive. However, the long plasma half-life of IgGs (up to three weeks in humans) causes hematological toxicities when used in radioimmunotherapy.

scFv-Fc Fusion Protein

Disclosed herein are re-engineered antibody fragments for diagnosis and treatment of diseases associated with the expression of PSCA. In some embodiments, engineering and reformatting the anti-PSCA A2 variant into a 110 kDa scFv-Fc2 fusion protein resulted in a fusion protein with improved affinity and optimized pharmacokinetics and biodistribution for imaging and radioimmunotherapy. The scFv-Fc fusion protein has a molecular weight that favors hepatobiliary clearance, reducing toxicity to the kidneys and intermediate-to-fast blood clearance due to point mutations in the FcRn binding site (single or double mutant) reducing hematological toxicity (i.e., “first point mutations”). In some embodiments, an additional point mutation (N297Q) removes the N-glycosylation site in the Fc region to further improve hepatic clearance (i.e., “second point mutation”).

The therapeutic index (TI) can be improved using engineered anti-PSCA A2 scFv-Fc2 antibody fragments (A2Fc2, t_{1/2} 74 h) with point mutations in the FcRn binding site (double mutant, A2DM, t_{1/2} 10 h) for rapid blood clearance and reduced bone marrow toxicity. Disclosed herein are novel variants with intermediate half-life to further improve the TI. In some embodiments, these novel variants also comprise antibody fragments (scFv-Fc fusion proteins) that have engineered human Immunoglobulin 2 Fc domains having a third point mutation K322A that results in reduced effector functions (ADCC and CDC).

Disclosed herein is a genetically engineered anti-prostate stem cell antigen (PSCA) scFv-Fc fusion protein. In various embodiments, the anti-PSCA scFv-Fc fusion protein comprises two peptides which form a homodimer, and each peptide comprises variable domains VH and VL of an anti-PSCA antibody, arranged in the order of VH-VL, engineered human IgG2 Fc domains, and a set of point mutations for rapid-medium clearance from circulation after administration to a subject. In some embodiments, the anti-PSCA scFv-Fc fusion protein comprises one or more first point mutations, such as H310A, H435Q, or both H310A and H435Q, to reduce half-life and hematological toxicity. In some embodiments, the anti-PSCA scFv-Fc fusion protein comprises a second point mutation, such as N297Q, to remove an N-glycosylation site in the Fc region to improve hepatic clearance. In some embodiments, the variable domains are connected by a glycine-rich linker to form an scFv. In certain embodiments, the anti-PSCA scFv-Fc fusion protein also comprises a third point mutation, K322A, which reduces effector functions (such as low antibody-dependent cellular cytotoxicity (ADCC) and complement-dependent cytotoxicity (CDC)).

The scFv-Fc are genetically aglycosylated for faster excretion through the liver. The anti-PSCA scFv-Fc disclosed herein bind specifically to human prostate stem cell antigen expressed on the cell surface of many cancers (prostate, pancreatic and bladder cancers) and can be used for detection, diagnosis, prognosis and treatment of PSCA-positive cancers.

In some embodiments, the optimized characteristics of the anti-PSCA scFv-Fc fusion protein disclosed herein result in inert antibody fragments with shorter serum half-life, but similar high-affinity bivalent PSCA binding compared to full-length antibodies. In some embodiments, the improved clearance and excretion through the hepatobiliary route facilitate imaging applications (immunoPET) at early time points and methods for treatment, e.g., radioimmunotherapy using radiometals, with the intention to reduce hematological and renal toxicity.

The anti-PSCA scFv-Fc fusion proteins disclosed herein can provide an improved TI for radioimmunotherapy of PSCA expressing tumors and are also useful for other tumor targeting applications in diagnosing and treating PSCA-positive tissues.

The term “antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (VL) and variable heavy chain (VH) refer to these light and heavy chains respectively.

In some embodiments, the scFv-Fc fusion protein disclosed herein comprises variable domains derived from anti-PSCA antibody A2, which is disclosed in U.S. Pat. No. 9,527,919, the content of which is incorporate by reference in its entirety and particularly with reference to its disclosure of antibody and antibody fragments such as VH, VL and CDR sequences, the structure of scFv and scFv-Fc, methods of making them, and their pharmaceutical compositions and formulations.

In some embodiments, the VH and VL domains of the scFv-Fc fusion protein disclosed herein have sequences substantially identical to those of the VH and VL domains of the A2 antibody disclosed in U.S. Pat. No. 9,527,919.

The term “substantially identical” in the context of two or more nucleic acid or amino acid sequences, refers to two or more sequences or subsequences that are the same or have a specified percentage of nucleotides or amino acid residues that are the same (i.e., about 80% identity, preferably at least 65%, 70%, 75%, 80%, 85%, 90%, 91%, 92%, 93%, 94%, 95%, 96%, 97%, 98%, 99%, or higher identity) over the referenced sequences or portions, when compared and aligned for maximum correspondence over a comparison window or designated region) as measured using a BLAST or BLAST 2.0 sequence comparison algorithms, or by manual alignment and visual inspection (see, e.g., NCBI web site or the like). The definition also includes sequences that have deletions and/or additions, as well as substitutions. Preferably, the sequence identity is at least 85%, 90%, 95%, 97%, 98% or 99% between two referenced domains. In some embodiments, the difference in sequence is just by one, two, three or four, or from five to 12, amino acids as to referenced sequence or domain. As described below, the preferred algorithms can account for gaps and the like. Preferably, identity exists over a region that is at least about 15 amino acids or nucleotides in length, or more preferably over a region that is 15-50 amino acids or nucleotides in length. In other embodiments, the identity may exist over a region that is at least about 50, 100, 150, 200, or more amino acids.

As to amino acid sequences, one of ordinary skill in the art will recognize that individual substitutions, deletions or additions to a nucleic acid, peptide, polypeptide, or protein sequence which alters, adds or deletes a single amino acid or a small percentage of amino acids in the encoded sequence is a “conservatively modified variant where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of this disclosure.

The following eight groups each contain amino acids that are conservative substitutions for one another: 1) Alanine (A), Glycine (G); 2) Aspartic acid (D), Glutamic acid (E); 3) Asparagine (N). Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M) (see, e.g., Creighton, Proteins (1984)).

In some embodiments, the scFv-Fc fusion protein is A2scFv-Fc2, which has a structure ordered as follows: VH-linker-VL-hinge-CH2-CH3, and having an amino acid sequence represented by SEQ ID NO: 2 below (possible sites for point mutations are bold/underlined:

EVQLVESGGG LVQPGGSLRL SCAASGFNIK DYYIHWVRQA
PGKGLEWVAW IDPEYGDSEF VPKFQGRATM SADTSKNTAY
LQMNSLRAED TAVYYCKTGG FWGRGTLVTV SSGGGGSGGG
GSGGSAQDIQ LTQSPSSLSA SVGDRVTITC SASSSVRFIH
WYQQKPGKAP KRLIYDTSKL ASGVPSRFSG SGSGTDFTLT
ISSLQPEDFA TYYCQQWGSS PFTFGQGTKV EIKVECPPCP
APPVAGPSVF LFPPKPKDTL MISRTPEVTC VVVDVSHEDP
EVQFNWYVDG MEVHNAKTKP REEQFNSTFR VVSVLTVVHQ
DWLNGKEYKC KVSNKGLPAP IEKTISKTKG QPREPQVYTL
PPSREEMTKN QVSLTCLVKG FYPSDIAVEW ESNGQPENNY
KTTPPMLDSD GSFFLYSKLT VDKSRWQQGN VFSCSVMHEA
LHNHYTQKSL SLSPGK

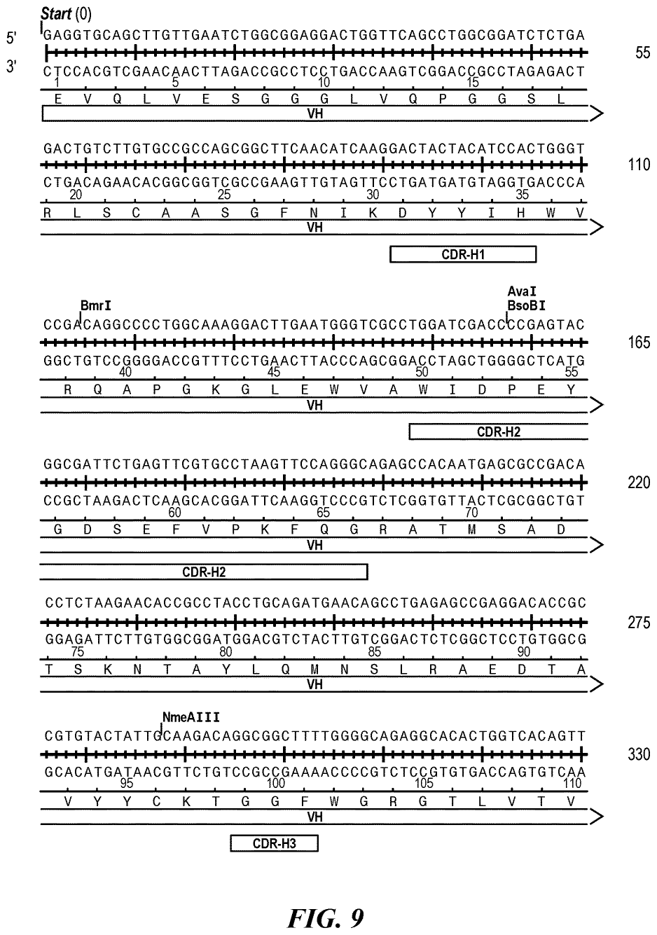

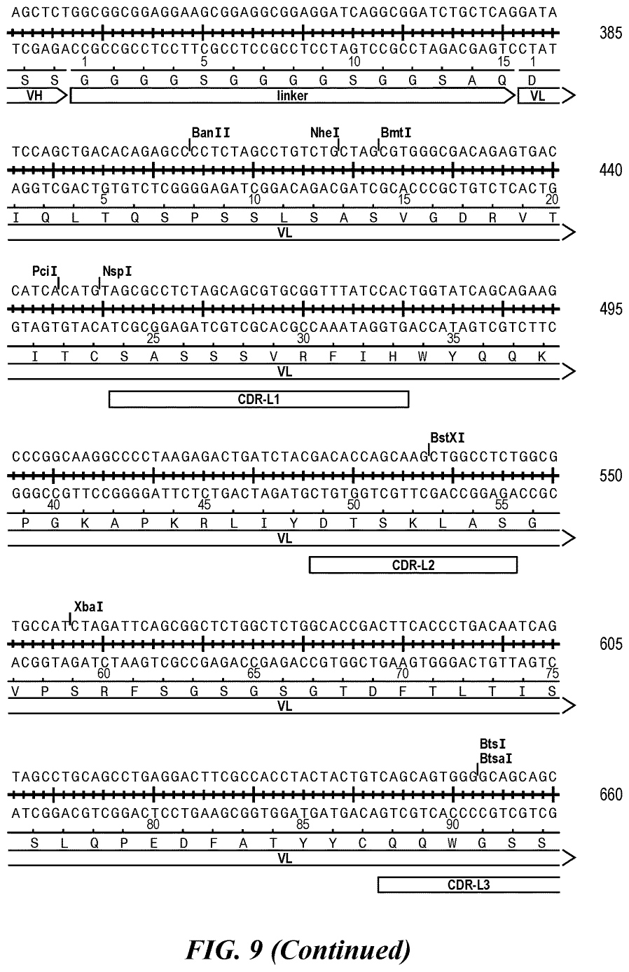

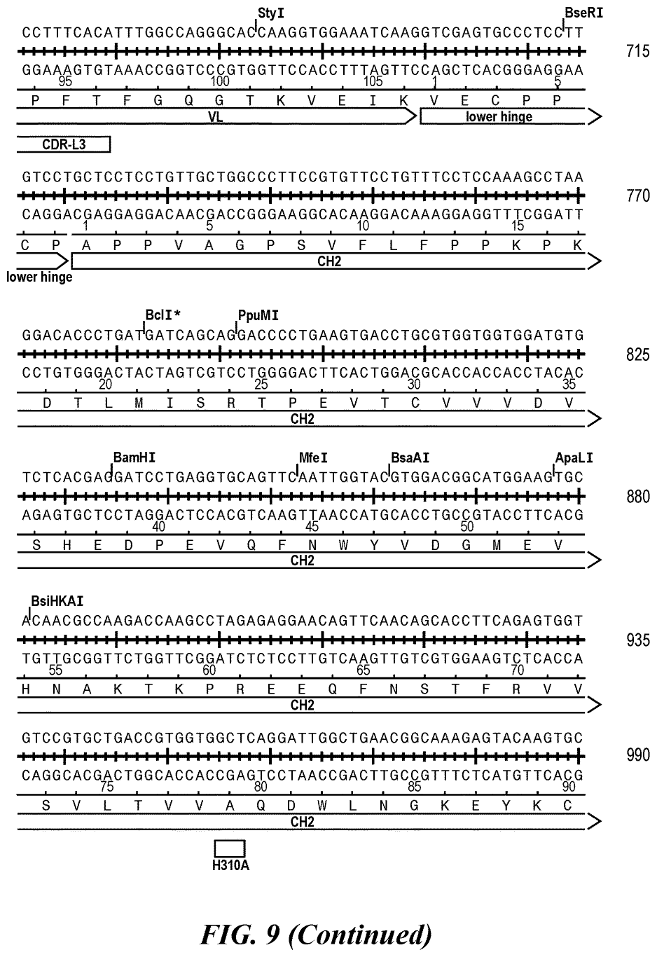

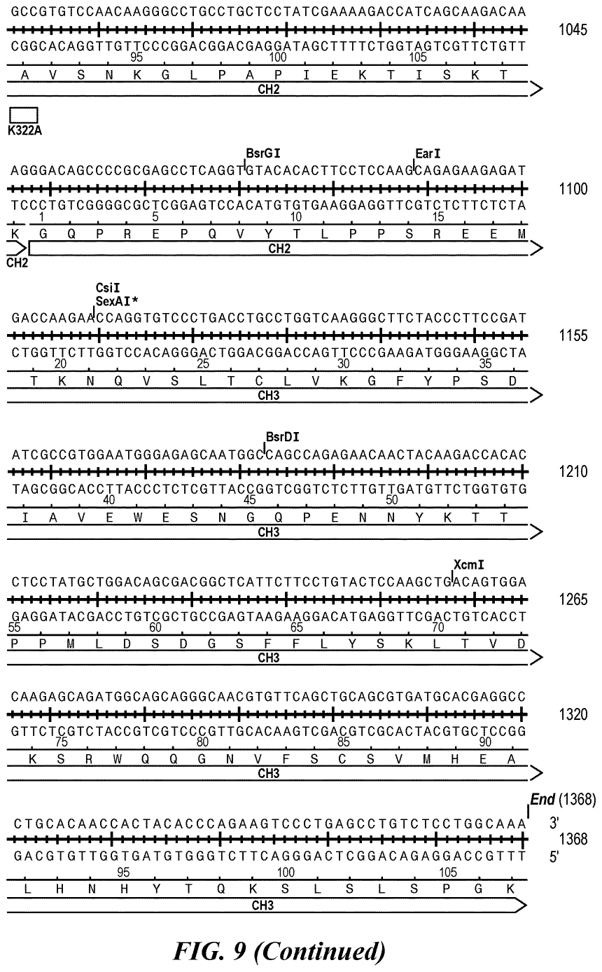

In some embodiments, the scFv-Fc fusion protein includes a single first point mutation at H310A (SM), no second point mutation, and a third point mutation at K322A (i.e., A2scFv-Fc2-SM w/K322A). The amino acid (SEQ ID NO:3) and nucleic acid (SEQ ID NO:4) sequences of A2scFv-Fc2-SM w/K322A are shown in FIG. 9. In other embodiments, the scFv-Fc fusion protein includes a single first point mutation at H310A (SM), no second point mutation, and no third point mutation (i.e., A2scFv-Fc2-SM w/o K322A). In other embodiments, the scFv-Fc fusion protein includes a single first point mutation at H310A (SM), a second aglycosylating point mutation at N297Q (aG), and no third point mutation (i.e., A2scFv-Fc2-SMaG w/o K322A). In other embodiments, the scFv-Fc fusion protein includes a single first point mutation at H310A (SM), a second aglycosylating point mutation at N297Q (aG), and a third point mutation at K322A (i.e., A2scFv-Fc2-SMaG w/K322A). In some embodiments, the first point mutation H310A is interchangeable with H435Q.

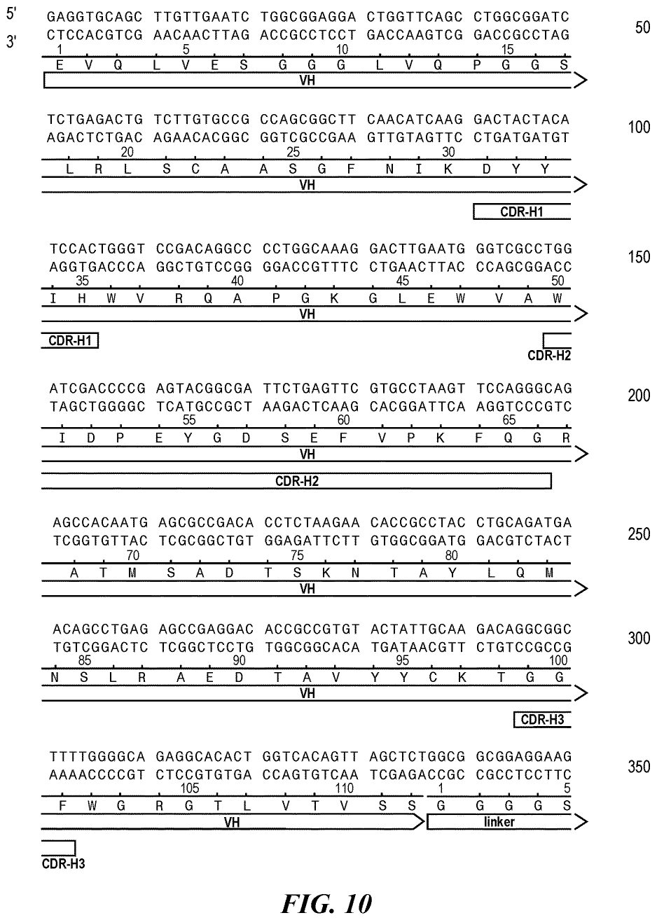

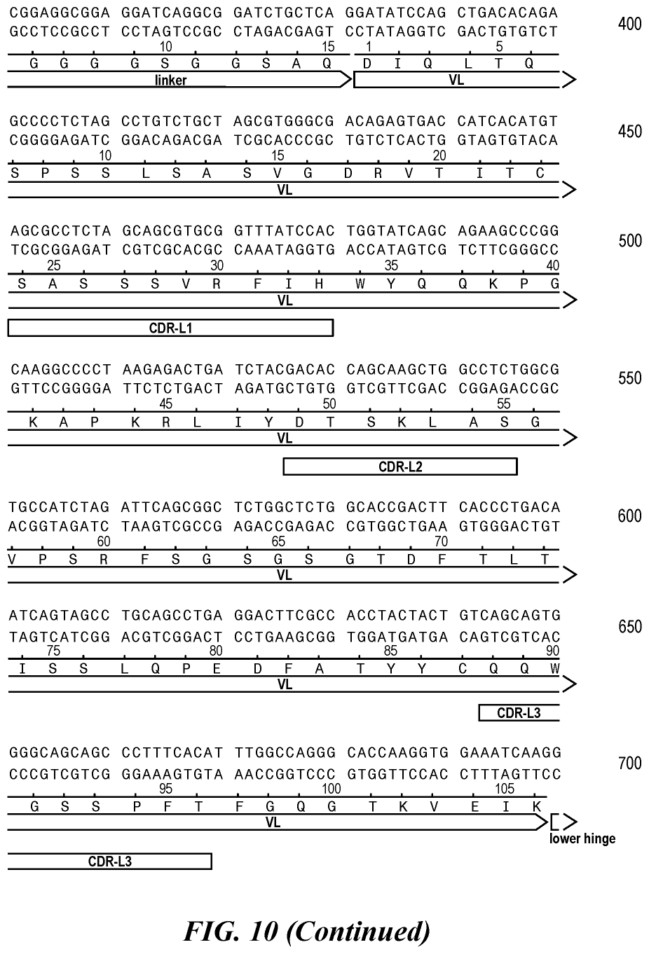

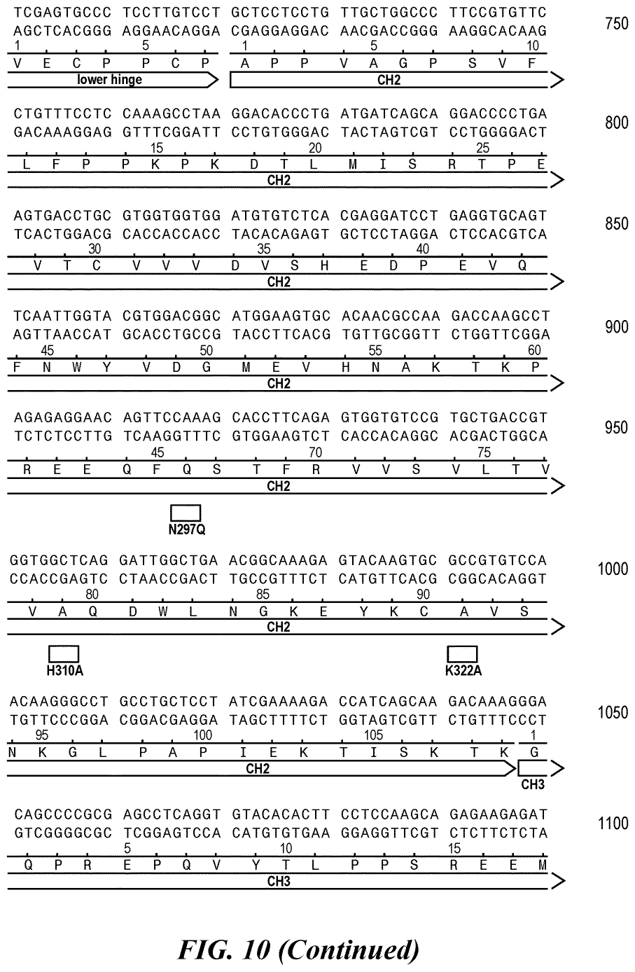

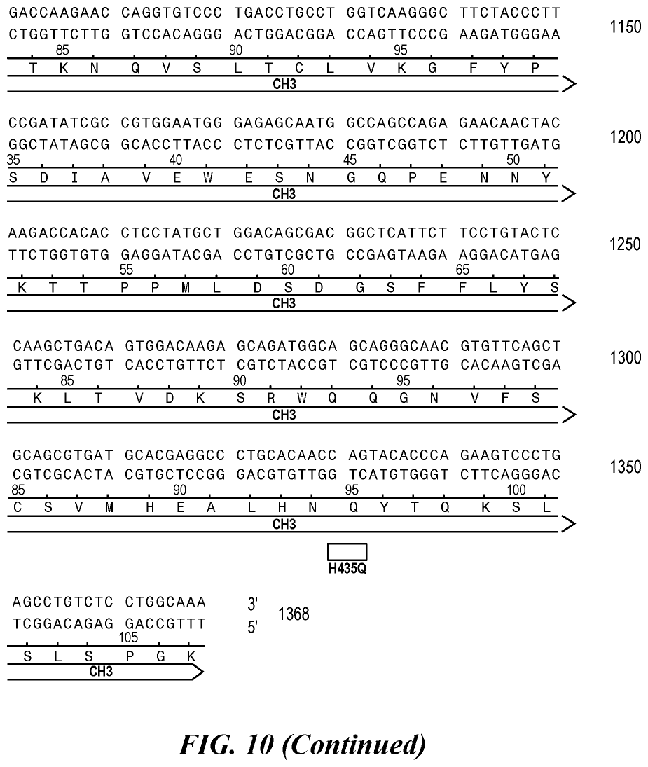

In other embodiments, the scFv-Fc fusion protein includes a double first point mutation at H310A and H435Q (DM), a second aglycosylating point mutation at N297Q (aG), and a third point mutation at K322A (i.e., A2scFv-Fc2-DMaG w/K322A). The amino acid (SEQ ID NO:5) and nucleic acid (SEQ ID NO:6) sequences of A2scFv-Fc2-DMaG w/K322A are shown in FIG. 10. In other embodiments, the scFv-Fc fusion protein includes a double first point mutation at H310A and H435Q (DM), no second point mutation, and no third point mutation (i.e., A2scFv-Fc2-DM w/o K322A). In other embodiments, the scFv-Fc fusion protein includes a double first point mutation at H310A and H435Q (DM); a second aglycosylating point mutation at N297Q (aG), and no third point mutation (i.e., A2scFv-Fc2-DMaG w/o K322A). In other embodiments, the scFv-Fc fusion protein includes a double first point mutation at H310A and H435Q (DM), a second aglycosylating point mutation at N297Q (aG), and a third point mutation at K322A (i.e., A2scFv-Fc2-DMaG w/K322A).

In some embodiments, the antibody fragments and/or domains of the disclosed fusion protein may be further modified, i.e., by the covalent attachment of any type of molecule to the antibody fragments and/or domains. For example, the modifications include, without limitation, glycosylation, acetylation, pegylation, phosphorylation, amidation, derivatization by known protecting/blocking groups, proteolytic cleavage, linkage to a cellular ligand or other protein, and the like. Any of numerous chemical modifications may be carried out by known techniques, including, but not limited to specific chemical cleavage, acetylation, formylation, metabolic synthesis of tunicamycin, etc. Additionally, the modifications include one or more non-natural amino acids.

The disclosed engineered fusion proteins recognize and specifically bind to PSCA with high affinity. These genetically engineered fusion proteins are tailored specifically for in vivo use for targeting and detection various cancers expressing PSCA, such as prostate cancer, pancreatic cancer and bladder cancer.

The phrase “specific binding” means that an engineered fusion protein binds to a particular target protein such as PSCA at least two times the background and more typically more than 10 to 100 times background. A variety of immunoassay formats may be used to select fusion proteins that specifically bind to PSCA. For example, solid-phase ELISA immunoassays are routinely used to select antibodies specifically binding to a target protein (see, e.g., Harlow & Lane, Using Antibodies, A Laboratory Manual (1998) for a description of immunoassay formats and conditions that can be used to determine specific immunoreactivity).

PSCA and its expression in cancer of the prostate, bladder, and pancreas is disclosed in U.S. Pat. No. 6,756,036 which is incorporated by reference in its entirety. The human PSCA translated amino acid sequence is represented by SEQ ID NO: 7 (UniProtKB-O43653), where the signal peptide is shown underlined and the propeptide is shown in bold and italic:

MAGLALQPGTALLCYSCKAQVSNEDCLQVENCTQLGEQCWTARIRAVGL
LTVISKGCSLNCVDDSQDYYVGKKNITCCDTDLCNASGAHALQPAAAIL
ALLPALGLLLWGPGQL

Also disclosed are polynucleotides comprising a nucleotide sequence encoding an antibody of the invention and fragments thereof. In certain embodiments, this disclosure provides an expression vector encoding the fusion protein disclosed herein. In certain embodiments, this disclosure provides polynucleotides encoding the fusion protein disclosed herein for use in gene therapy or in vivo administration.

In certain embodiments, the fusion protein disclosed herein is conjugated to an “effector” moiety. The effector moiety can be various molecules, including labeling moieties such as detectable markers including radioactive labels or fluorescent labels, or can be a therapeutic moiety. Such effector moieties include but are not limited to, a cytotoxic agent, an anti-tumor drug, a toxin, a radioactive agent, a cytokine, a second protein, an antibody, or an enzyme. Further, the fusion protein disclosed herein can be linked to an enzyme that converts a prodrug into a cytotoxic agent.

Examples of cytotoxic agents include, but are not limited to ricin, doxorubicin, daunorubicin, TAXOL, ethidium bromide, mitomycin, etoposide, tenoposide, vincristine, vinblastine, colchicine, dihydroxy anthracin dione, actinomycin D, diptheria toxin, Pseudomonas exotoxin (PE) A, PE40, abrin, and glucocorticoid and other chemotherapeutic agents, as well as radioisotopes. Suitable detectable markers include, but are not limited to, a radioisotope, a fluorescent compound, a bioluminescent compound, chemiluminescent compound, a metal chelator or an enzyme. The second protein can include, but is not limited to, an enzyme, lymphokine, oncostatin or toxin. Suitable toxins include doxorubicin, daunorubicin, TAXOL, ethidium bromide, mitomycin, etoposide, tenoposide, vincristine, vinblastine, colchicine, dihydroxy anthracin dione, actinomycin D, diptheria toxin, Pseudomonas exotoxin (PE) A, PE40, ricin, abrin, glucocorticoid and radioisotopes.

Techniques for conjugating therapeutic agents to constructs according to the invention are well known (see, e.g., Amon et al., “Monoclonal Antibodies For Immunotargeting Of Drugs. In Cancer Therapy,” in Monoclonal Antibodies And Cancer Therapy, Reisfeld et al. (eds.), pp. 243-56 (Alan R. Liss, Inc. 1985) Hellstrom et al., “Antibodies For Drug Delivery’ in Controlled Drug Delivery (2nd Ed.), Robinson et al. (eds.), pp. 623-53 (Marcel Dekker, Inc. 1987); Thorpe, “Antibody Carriers Of Cytotoxic Agents. In Cancer Therapy: A Review” in Monoclonal Antibodies 84: Biological And Clinical, Applications, Pinchera et al. (eds.), pp. 475-506 (1985) and Thorpe et al., “The Preparation And Cytotoxic Properties Of Antibody-Toxin Conjugates. Immunol. Rev., 62:119-58 (1982)).

### Pharmaceutical Compositions

Also disclosed is a pharmaceutical composition comprising an effective amount of one or more anti-PSCA scFv-Fc fusion proteins or one or more anti-PSCA scFv-Fc fusion protein and effector moiety conjugates disclosed herein. The pharmaceutical composition can further comprise one or more additional therapeutic agents, and/or one or more pharmaceutically acceptable carriers, excipients, and stabilizers.

Acceptable carriers, excipients or stabilizers can be acetate, phosphate, citrate, and other organic acids; antioxidants (e.g., ascorbic acid) preservatives low molecular weight polypeptides; proteins, such as serum albumin or gelatin, or hydrophilic polymers as such polyvinylpyllolidone; and amino acids, monosaccharides, disaccharides, and other carbohydrates including glucose, mannose, or dextrins; chelating agents; and ionic and non-ionic surfactants (e.g., polysorbate); salt-forming counter-ions such as sodium; metal complexes (e.g., Zn-protein complexes); and/or non-ionic surfactants. The fusion protein, or the conjugate can be formulated at a concentration of between 0.5-200 mg/ml, or between 10-50 mg/ml.

Additional therapeutic agents include, for example, chemotherapeutic agents, cytotoxic agents, cytokines, growth inhibitory agents, anti-hormonal agents, and radionuclide including alpha- or beta-emitting radioisotopes such as At-211, Ac-225, Cu-67, Y-90, Lu-177, and I-131. The therapeutic agents may also be prepared as sustained-release preparations (e.g., semi-permeable matrices of solid hydrophobic polymers (e.g., polyesters, hydrogels (for example, poly(2-hydroxyethyl-methacrylate), or poly(vinylalcohol)), polylactides. The fusion proteins, or conjugates may also be entrapped in microcapsules prepared, for example, by coacervation techniques or by interfacial polymerization, for example, hydroxymethylcellulose or gelatin microcapsules and poly-(methylmethacylate) microcapsules, respectively, in colloidal drug delivery systems (for example, liposomes, albumin microspheres, microemulsions, nano-particles and nanocapsules) or in macroemulsions. In certain embodiments, the therapeutic agent is conjugated to the anti-PSCA scFv-Fc fusion protein.

The term “effective amount” “therapeutically effective amount” or “therapeutically effective dose” as used herein refers to an amount of a composition that produces a desired effect. An effective amount of a fusion protein, a conjugate, or a pharmaceutical composition may be used to produce a therapeutic effect in a subject, such as preventing or treating a target condition, alleviating symptoms associated with the condition, or producing a desired physiological effect. In such a case, the effective amount is a “therapeutically effective amount,” “therapeutically effective concentration” or “therapeutically effective dose.” The precise effective amount or therapeutically effective amount is an amount of the fusion protein, the conjugate, or the pharmaceutical composition that will yield the most effective results in terms of efficacy of treatment in a given subject. This amount will vary depending upon a variety of factors, including but not limited to the characteristics of the active agent (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. Further, an effective or therapeutically effective amount may vary depending on whether the fusion protein, the conjugate, or the pharmaceutical composition is administered alone or in combination with another composition, drug, therapy or other therapeutic method or modality. One skilled in the clinical and pharmacological arts will be able to determine an effective amount or therapeutically effective amount through routine experimentation, namely by monitoring the subject's response to administration of the fusion protein, the conjugate, or the pharmaceutical composition and adjusting the dosage accordingly. For additional guidance, see Remington: The Science and Practice of Pharmacy, 21 st Edition, Univ. of Sciences in Philadelphia (USIP), Lippincott Williams & Wilkins, Philadelphia, PA, 2005, which is hereby incorporated by reference as if fully set forth herein.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The pharmaceutical composition can, if desired, also contain other compatible therapeutic agents.

Preferred pharmaceutical preparations deliver one or more fusion proteins, or conjugates disclosed herein, optionally in combination with one or more chemotherapeutic agents or immunotherapeutic agents, in a sustained release formulation. The fusion proteins, or conjugates disclosed herein may be administered therapeutically as a sensitizing agent that increases the susceptibility of tumor cells to other cytotoxic cancer therapies, including chemotherapy, radiation therapy, immunotherapy and hormonal therapy.

Use of the scFv-Fc Fusion Protein

The scFv-Fc fusion proteins disclosed herein can be used in the diagnosis, prognosis and treatment of cancers which overexpress PSCA, for example, prostate, pancreatic and bladder cancers. Thus, this disclosure also relates to a method of diagnosing, prognosing, or treating a cancer overexpressing PSCA by administering an effective amount of the scFv-Fc fusion proteins or the pharmaceutical compositions disclosed herein to a subject. In certain embodiments the method is applied to hormone refractory or therapy resistant cancers. In certain embodiments the method is applied to metastatic cancers.

“Treating” or “treatment” of a condition may refer to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof. Treatment may also mean a prophylactic or preventative treatment of a condition.

The fusion proteins, conjugates and pharmaceutical compositions disclosed herein can be administered for therapeutic or prophylactic treatments. In therapeutic applications, compositions are administered to a patient suffering from a disease (e.g., cancer) in a “therapeutically effective dose.” Amounts effective for this use will depend upon the severity of the disease and the general state of the patient's health. Single or multiple administrations may be administered depending on the dosage and frequency as required and tolerated by the patient. A “patient’ or “subject’ for the purposes of the present invention includes both humans and other animals, particularly mammals. Thus, the methods are applicable to both human therapy and veterinary applications. Other known cancer therapies can be used in combination with the methods of the invention. For example, the fusion protein, the conjugate, or the pharmaceutical composition for use according to this disclosure may also be used to target or sensitize a cell to other cancer therapeutic agents such as 5FU, vinblastine, actinomycin D, cisplatin, methotrexate, and the like.

In other embodiments, the methods may be practiced together with other cancer therapies (e.g., radical prostatectomy), radiation therapy (external beam or brachytherapy), hormone therapy (e.g., orchiectomy, LHRH-analog therapy to suppress testosterone production, anti-androgen therapy), or chemotherapy. Radical prostatectomy involves removal of the entire prostate gland plus some surrounding tissue. This treatment is used commonly when the cancer is thought not to have spread beyond the tissue. Radiation therapy is commonly used to treat prostate cancer that is still confined to the prostate gland or has spread to nearby tissue. If the disease is more advanced, radiation may be used to reduce the size of the tumor. Hormone therapy is often used for patients whose prostate cancer has spread beyond the prostate or has recurred. The objective of hormone therapy is to lower levels of the male hormones, androgens and thereby cause the prostate cancer to shrink or grow more slowly. Luteinizing hormone-releasing hormone (LHRH) agonists decrease the production of testosterone. These agents may be injected either monthly or longer. Two such analogs are leuprolide and Goserelin. Anti-androgens (e.g., flutamide, bicalutamide, and nilutamide) may also be used. Total androgen blockade refers to the use of anti-androgens in combination with orchiectomy or LHRH analogs. Chemotherapy is an option for patients whose prostate cancer has spread outside of the prostate gland and for whom hormone therapy has failed. It is not expected to destroy all of the cancer cells, but it may slow tumor growth and reduce pain. Some of the chemotherapy drugs used in treating prostate cancer that has returned or continued to grow and spread after treatment with hormonal therapy include doxorubicin (Adriamycin), estramustine, etoposide, mitoxantrone, vinblastine, and paclitaxel. Two or more drugs are often given together to reduce the likelihood of the cancer cells becoming resistant to chemotherapy. Small cell carcinoma is a rare type of prostate cancer that is more likely to respond to chemotherapy than to hormonal therapy.

The combined administrations contemplate co-administration, using separate formulations or a single pharmaceutical formulation, and consecutive administration in either order, wherein preferably there is a time period while both (or all) active agents simultaneously exert their biological activities.

Treatment will generally involve the repeated administration of the fusion proteins or the pharmaceutical compositions via an acceptable route of administration such as intravenous injection (IN), at an effective dose. Dosages will depend upon various factors generally appreciated by those of skill in the art, including without limitation, the type of cancer and the severity, grade, or stage of the cancer, the binding affinity and half-life of the fusion proteins used, the desired steady-state concentration level, frequency of treatment, and the influence of chemotherapeutic agents or other therapeutic agents used in combination with the treatment method of the invention. Typical doses may range from about 0.1 to 100 mg/kg. Doses in the range of 10-500 mg of the fusion proteins per week may be effective and well tolerated, although even higher weekly doses may be appropriate and/or well tolerated. In certain embodiments, the administration schedule is weekly or every 2-4 weeks. The principal determining factor in defining the appropriate dose is the amount of a particular agent necessary to be therapeutically effective in a particular context. Repeated administrations may be required in order to achieve tumor inhibition or regression. Initial loading doses may be higher. The initial loading dose may be administered as an infusion. Periodic maintenance doses may be administered similarly, provided the initial dose is well tolerated.

In therapeutic use for the treatment of cancer, the dosage of the fusion proteins, or the conjugates may be varied depending upon the requirements of the patient, the severity of the condition being treated, and the active agent being employed. For example, dosages can be empirically determined considering the type and stage of cancer diagnosed in a particular patient. The dose administered to a patient should be sufficient to affect a beneficial therapeutic response in the patient over time. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

Direct administration of the fusion proteins is also possible and may have advantages in certain contexts. For example, for the treatment of bladder carcinoma, the fusion proteins or the pharmaceutical compositions may be injected directly into the bladder.

The fusion proteins, conjugates and pharmaceutical compositions disclosed herein can be administered to a subject using various known methods, such as intravenous administration, e.g., as a bolus or by continuous infusion over a period of time, by intramuscular, intraperitoneal, intracerobrospinal, subcutaneous, intraarticular, intrasynovial, intrathecal, oral, topical, or inhalation routes. Intravenous or subcutaneous administration is preferred. The administration may be local or systemic.

The pharmaceutical compositions for administration will commonly comprise a fusion protein, or a conjugate as described herein dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patients' needs.

The pharmaceutical compositions can be administered in a variety of dosage forms depending upon the method of administration. For example, unit dosage forms suitable for oral administration include, but are not limited to, powder, tablets, pills, capsules and lozenges. It is recognized that when administered orally, the pharmaceutical composition should be protected from digestion. This is typically accomplished either by complexing the active agent with a composition to render them resistant to acidic and enzymatic hydrolysis, or by packaging the active agents in an appropriately resistant carrier, such as a liposome or a protection barrier. Means of protecting agents from digestion are well known in the art.

Methods of Detecting Cancer and/or Tumor Imaging

In another aspect, disclosed herein is a method of detecting cancer or tumor imaging in vivo through administration of the anti-PSCA scFv-Fc fusion protein disclosed herein. In one embodiment, provided herein is a method of imaging a cancer cell in vivo, the method comprising administering a labeled anti-PSCA scFv-Fc fusion protein to a mammal and imaging the fusion protein in vivo. The mammal includes without limitation, a mouse, rat, hamster, rabbit, pig, human, and the like.

A “label” or a “detectable moiety” or “detectable marker” is a composition detectable by spectroscopic, photochemical, biochemical, immunochemical, chemical, or other physical means. In some aspects, the detectable marker is detected by intraoperative imaging. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

In some embodiments, the detectable moiety is an infrared or near-infrared detectable moiety that is detectable by intraoperative imaging. The infrared or near-infrared detectable moiety may be fluorescent in the rage of 700 nm-900 nm. For example, the detectable moiety may be a heptamethine indocyanine such as indocyanine green (ICG). In some aspects, the detectable moiety may be IRDye800CW, IR800 free acid, OTL-38, folate-heptamethine indocyanine, HaloTag, SNAP-tag, FNIR-Tag-766, or any other infrared or near-infrared detectable moiety described in S. Usama et al. 145 J. Am. Chem. Soc. 14647 (2023) and U.S. Patent Publication No. 2023/0241253; the detectable moieties described in these publications are incorporated by reference herein. In some aspects, the detectable moiety is IRDye800CW.

Methods of in vivo imaging are well known in the art and include without limitation, magnetic resonance imaging (MRI), nuclear magnetic resonance (NMR) (R. Weissleder, 1999, Radiology 212:609-14), computerized axial tomography (CAT) scan, cooled charged coupled device (CCD) camera optical imaging (Honigman, et al., 2001 Mol. Ther. 4:239-249), bioluminescent optical imaging (P R Contag, et al., 1998 Nat. Med. 4:245-247), position emission tomography (PET) (M E Phelps, 1991 Neurochemical Research 16:929-994: J G Tuvajev, et al., 1998 Cancer Res 58:4333-4341), single photon emission computed tomography (J G. Tuvajev, et al., 1996 Cancer Res. 45:4087-4095), microPET (reviewed in Mcveigh, 2006, Circ. Res. 98:879-86), and the like.

The following examples are intended to illustrate various embodiments of the invention. As such, the specific embodiments discussed are not to be constructed as limitations on the scope of the invention. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### EXAMPLES

### Example 1: Design and Purification of A2 scFv-Fc2 SM and A2 scFv-Fc2 DMaG

### Materials and Methods

Design and cloning of A2 scFv-Fc2 SM and A2 scFv-Fc2 DMaG antibody fragments The single-chain variable fragment-crystallizable fragment fusion protein (scFv-Fc) consists of the scFv fused to the Fc region of human IgG2. The scFv in VH-VL sequence is connected by a 15 amino acid glycine-rich linker (G4S) 2-GGSAQ. The human gamma 2 Fc domain contains a truncated hinge followed by CH2 and CH3 domains and a point mutation in the CH2 (K322A) resulting in reduced effector functions (ADCC, CDC).

The point mutation H310A in the FcRn binding region of the Fc was introduced for construct A2scFv-Fc2 SM (single mutant) for intermediate blood half-life. The A2scFc-Fc2 DMaG (double mutant aglycosylated) contains two first point mutations in the Fc region (H310A, H435Q) that modulate FcRn binding. The second point mutation at asparagine 297 (N297Q) to remove the N-glycosylation site was introduced by site-directed mutagenesis using primers aG SDM F (27 bp): 5′-GAACAGTTCCAAAGCACCTTCAGAGTG-3′ and aG SDM R (26 bp): 5′-GAAGGTGCTTTGGAACTGTTCCTCTC-3′ and the Fusion Site-Directed Mutagenesis Kit (Thermo Scientific).

The synthetic codon-optimized DNA (in plasmid pMA-T, Invitrogen by ThermoFisher Scientific) encoding for the two A2scFv-Fc constructs were sub-cloned into the mammalian expression vector pSecTag2A (AgeI) using the restriction sites AgeI and NotI. The resulting plasmids were transfected into mammalian cell line FS293-F (FreeStyle™ 293-F cells; Gibco, ThermoFisher Scientific) using Lipofectamine™ 3000 (ThermoFisher Scientific). Stable cell pools were generated by Zeocin™ Selection Reagent (ThermoFisher Scientific) selection and tested for protein expression by Western blot.

Protein production: Zeocin selected stable cell pools were expanded into Nunc TripleFlask and growth medium was replaced with serum-free medium (Opti-MEM®, ThermoFisher Scientific) for protein expression. Cell culture supernatant was collected every 3-4 days for two weeks.

Protein purification: Recombinant anti-PSCA scFv-Fc2 fusion proteins were purified from cell culture supernatants using an Akta™ chromatography system (GE Healthcare) with column CaptureSelect FcXL (ThermoScientific). Columns were equilibrated using 5 column volumes of binding buffer (20 mM sodium phosphate pH 7.0). Prior to loading supernatants were concentrated and adjusted to pH 8.0-9.0 by adding 1/10 volume of 1M TrisHCI pH 9.0. Unbound protein was removed by washing the column with binding buffer. Bound protein was eluted with 100 mM citric acid pH 3.0, neutralized by adding 1/10 volume 1M TrisHCI pH 9.0 and protein containing fractions were dialyzed against PBS.

Biochemical characterization: Purified proteins were analyzed by SDS-PAGE, Western blot and size exclusion chromatography (Superdex®200 Increase 10/300 GL, ThermoFisher Scientific) for purity, integrity and apparent molecular weight.

Specificity and affinity of antigen binding was further evaluated by performing saturation binding on immobilized antigen in ELISA. Recombinant human PSCA-mouse Fc fusion protein (hPSCA-mFc, produced in house) was immobilized (1 μg/mL, 100 L/well in PBS overnight at 4° C.). Serial dilutions of anti-PSCA scFvFc's (1 μM-0.1 μM) were incubated for 1 h at RT and after washing bound antibody fragments were detected using goat anti-human IgG-Fc-AP antibody (1:10,000) and pNPP substrate (alkaline phosphatase yellow, liquid substrate, Sigma-Aldrich P7998). The enzymatic reaction was stopped using 50 μL 2M NaOH and absorbance was red at 405 nm in an SpectraMax M2 microplane reader. Specific binding was fitted using the “one site-specific binding” equation to determine the K_{d} (ligand concentration that binds half the antigen sites at equilibrium) and Bₘₐₓ (maximum number of binding sites).

Cell binding (flow cytometry): Murine prostate cancer cells (RM9 and RM9-hPSCA, 1×10⁶ cells/100 μL PBS, 1% FBS) were incubated with A2SM or A2DMaG (1 or 10 μg/mL) for 0.5 h at 4° C. After washing, bound antibody was detected using a goat anti-human IgG-AlexaFluor 647 secondary antibody and cells were analyzed on a BD LSRFortessa X-20 Cell Analyzer.

For saturation binding curves, cells (0.5×10⁶ cells/500 μL PBS, 1% FBS) were incubated with serial dilution (1:3 dilution, 1 μM-0.63 μM) for 2 h at 4° C. on a rotator. Cells were washed, incubated with secondary antibody and analyzed as described above.

DFO-conjugation and radiolabeling (zirconium-89): Anti-PSCA A2SM and A2DMaG were incubated with p-SCN-Bn-Deferoxamine (SCN-DFO, Macrocyclics, B-705) at 5-fold molar excess (pH 9.0, 2 h, room temperature). Excess SCN-DFO was removed using Micro Bio-Spin chromatography columns (BioRad) preconditioned with PBS. Successful conjugation was confirmed by SDS-PAGE (Coomassie-stained) and SEC (Superdex 200) analysis.

[⁸⁹Zr]Zr-oxalate (3D Imaging LLC) was neutralized (0.45 vol 2 M Na₂CO₃, 2.5 vol 1M HEPES) and added to the DFO-conjugated protein (0.185-0.278 MBq/5-7.5 μCi/μg) for 1 h at room temperature. Radiolabeled protein was purified using Micro Bio-Spin chromatography columns (BioRad). Labeling efficiency and radiochemical purity were determined by ITLC with 20 mM citric acid (pH 5.0) as solvent.

ImmunoPET/CT (in vivo): Mice (hPSCA KI x C57BL6/J) with RM9-hPSCA or RM9 subcutaneous tumors, were injected via the tail vein with 10 μg (1.3-2.6 MBq/35-70 μCi) of ⁸⁹Zr-A2SM. Mice were anesthetized with 2-3% isoflurane and 10-minute static PET scans followed by a 1-minute standard CT scan were acquired on the B-CUBE (PET) an X-CUBE (CT) (Molecubes Modular Benchtop Imaging) at indicated time points post injection (p.i.). Images were reconstructed using an 3D-OSEM MAP algorithm and are presented as whole-body maximum intensity projection (MIP) PET/CT overlays using VivoQuant software.

### Results

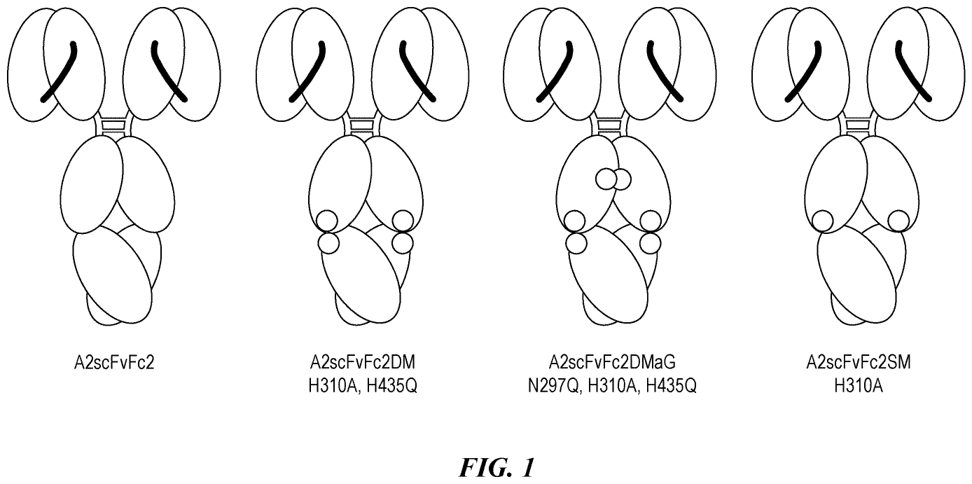

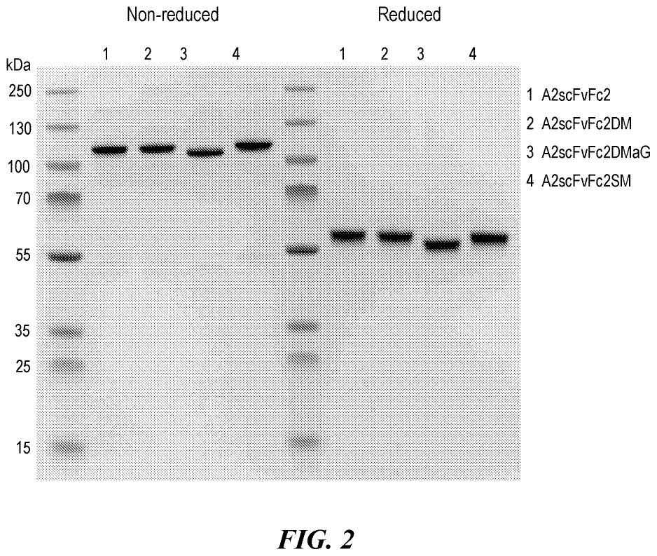

As illustrated in FIG. 1, Novel scFv-Fc fragments based on anti-PSCA antibody fragment A2 were designed by changing the order of the variable domains to VH-VL, connected by a 15 amino acid glycine-rich linker ((G4S) 2-GGSAQ) followed by a human immunoglobulin 2 (IgG2) truncated hinge and fragment crystallizable (Fc) comprising a point mutation (K322A) that reduced C1q binding and thereby CDC. The double mutant aglycosylated (A2DMaG) derivative contains two first point mutations replacing histidine residues involved in FcRn binding with alanine or glutamine, respectively (H310A/H435Q) and a second point mutation removing the conserved N-glycosylation site (N297Q). The single mutant (A2SM) contains only the H310A first point mutation but could also be combined with the aG second point mutation.

Codon optimized genes encoding the scFv-Fc proteins were subcloned into mammalian expression vector (pSECTag2A), transfected into FreeStyle 293-F cells and stable cell pools selected.

Recombinant scFv-Fc fusion proteins were purified from mammalian cell culture supernatant. SDS-Page analysis of the purified protein under non-reducing conditions showed a single band with an apparent molecular mass of approximately 110 kDa (calculated M_{W} 99.65 kDa for A2Fc2 and 99.5 kDa for A2DM, and 99.5 kDa A2SM) consistent with the glycosylated (Asn-297, C_{H}2) homodimer (FIG. 2). The A2DMaG protein is not glycosylated and migrates with an apparent molecular weight of approximately 100 kDa (calculated M_{W} 99.5 kDa for A2DMaG. Under reducing conditions the disulfide bridges in the hinge are broken and the monomeric proteins migrate at approximately 55 kDa while the A2DMaG migrates slightly smaller, confirming that the protein is not glycosylated.

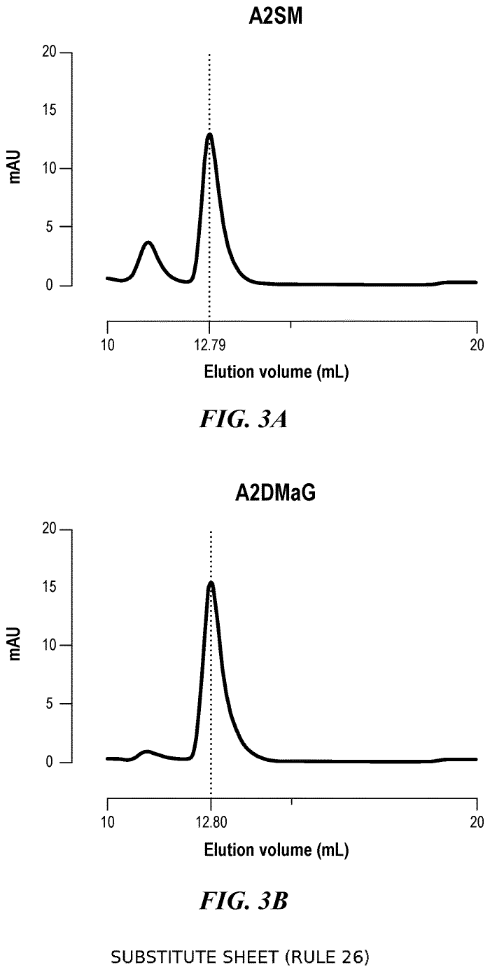

Purity, integrity and apparent molecular weight of the purified anti-PSCA scFv-Fc's were confirmed by size exclusion chromatography (FIG. 3). Both A2SM and A2DMaG eluted as major single peak (93-95% AUC) at elution volumes (12.79 and 12.80 mL, respectively) concordant with the calculated molecular weight of approximately 110 kDa (A2SM) and 100 kDa (A2DMaG).

### Example 2: Antigen-Specific Binding to Recombinant hPSCA

Unless otherwise specified below, the materials and methods are the same as those disclosed above in Example 1.

The purified proteins were tested for specific binding to recombinant antigen by ELISA on immobilized human PSCA-mouse Fc protein. Dose-dependent binding was detected for wells coated with hPSCA-mFc and no non-specific binding was detected in BSA coated control wells.

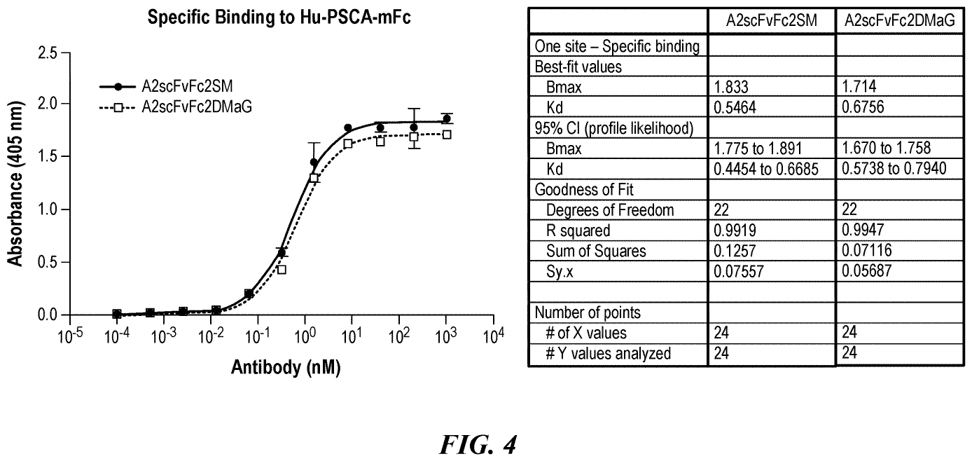

Saturation binding curves (in duplicates, n=3) on hPSCA-mFc were used to calculate the half-maximal binding (EC50=K_{d}) (FIG. 4). The resulting K_{d} for A2SM (0.59±0.03 nM) and for A2DMaG (0.80±0.11 nM) were in the same sub-nanomolar range as the previous A2Fc2 (0.5±0.1 nM) and A2DM (0.8±0.2 nM) confirming that the re-engineered new variants retain antigen specificity and affinity.

### Example 3: Specific Binding to PSCA-Expressing Cells and Cell Binding Affinity

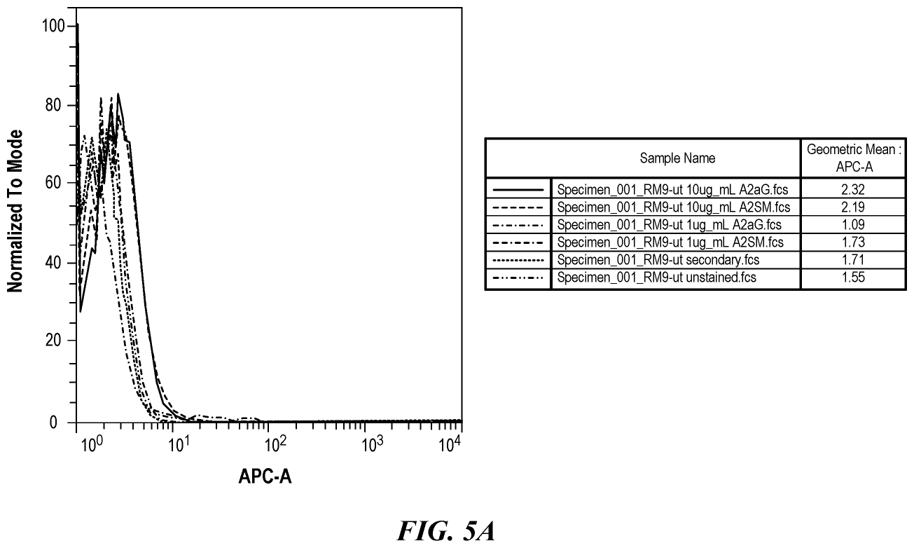

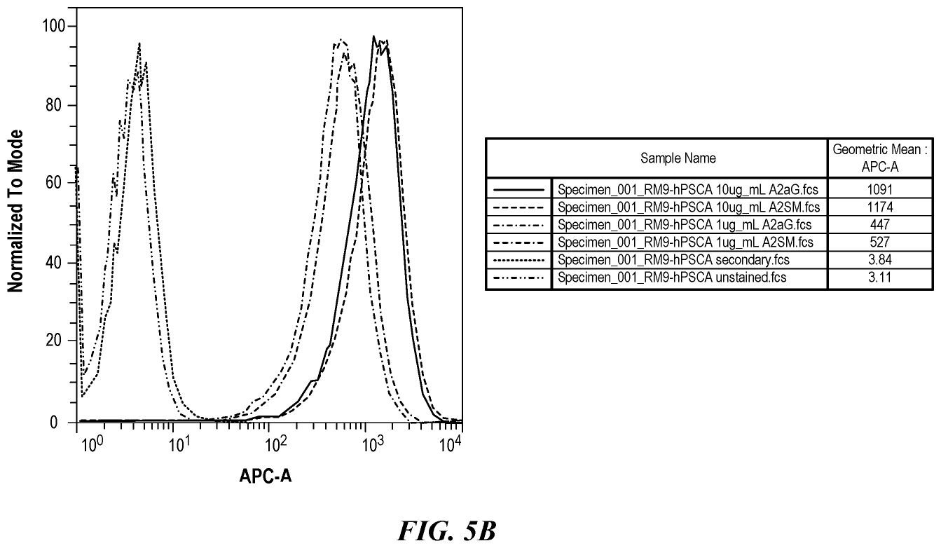

Binding of the novel anti-PSCA antibody fragments A2SM and A2DMaG to PSCA expressed on the cell surface of mouse prostate cancer cells was analyzed by flow cytometry. Specific binding was confirmed on RM9-hPSCA cells and no binding was seen with antigen negative RM9 cells (FIG. 5).

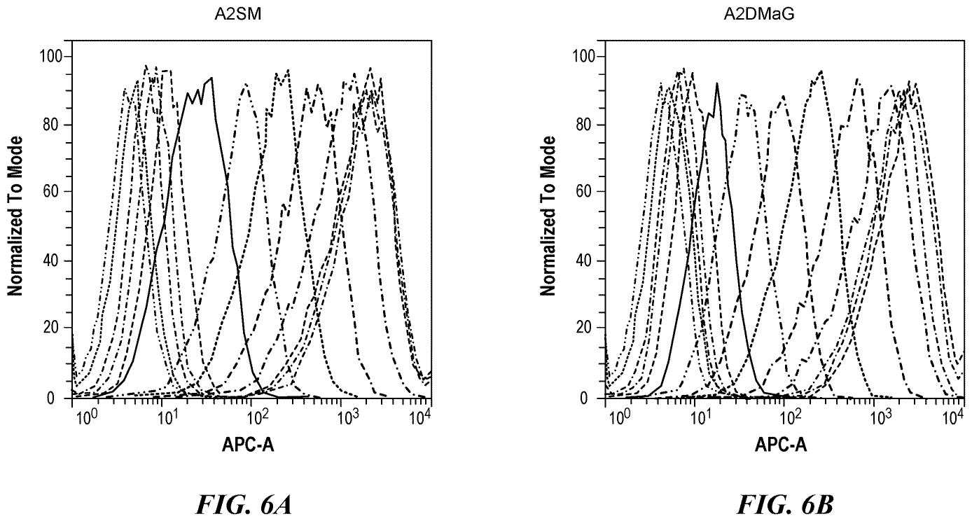

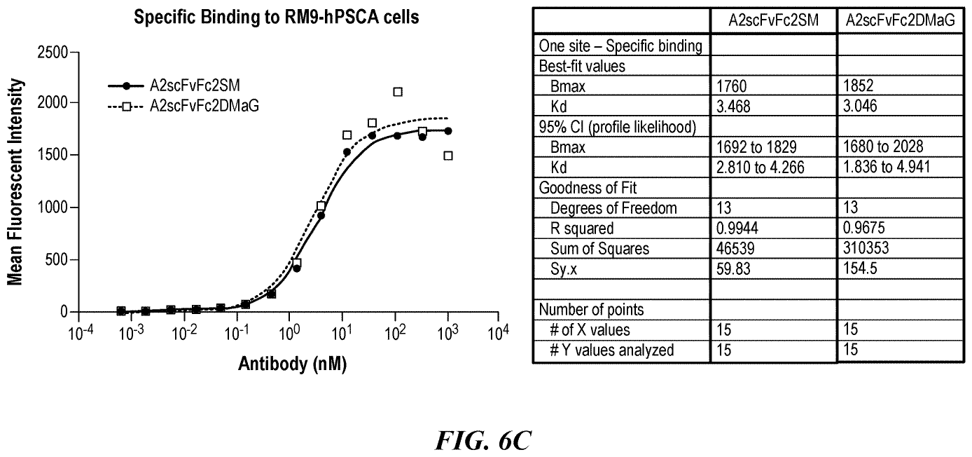

The apparent cell binding affinity was evaluated by incubating RM9-hPSCA cells with serial dilutions of anti-PSCA antibody fragments until binding reached equilibrium (0.5×10⁶ cells/0.5 mL, 2 h at 4° C., rotating). The resulting saturation binding curves were fitted (one-site specific binding, GraphPad Prism) to determine K_{d} and Bₘₐₓ from four independent experiments. A2SM has a K_{d} of 3.74±0.54 nM and A2DMaG show a K_{d} of 3.28±0.59 nM. (FIG. 6)

### Example 4: Radiolabeling

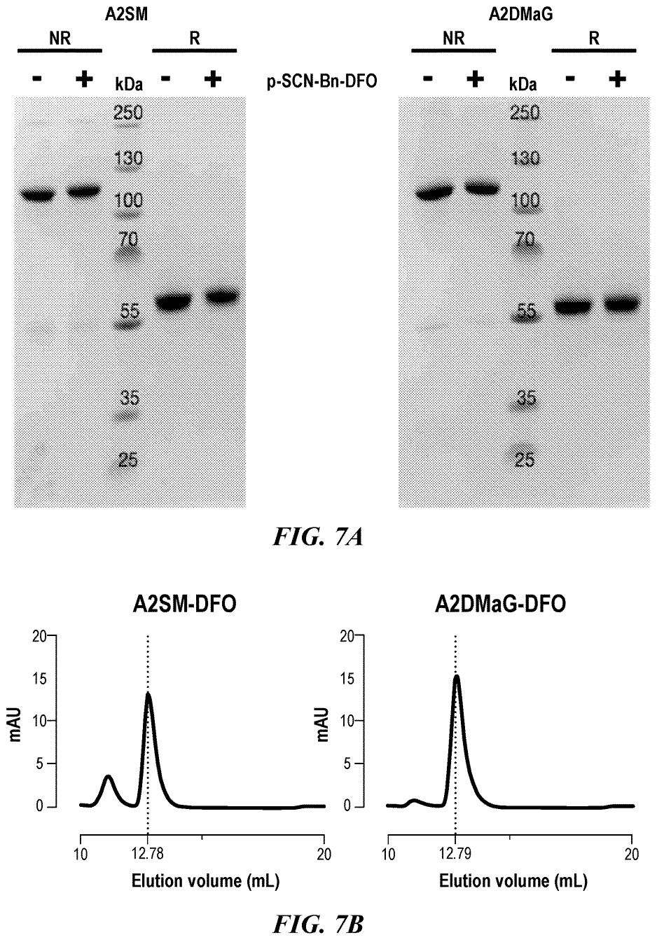

The anti-PSCA A2scFv-Fc SM and DMaG antibody fragments were conjugated with the bifunctional chelator p-SCN-Bn-deferoxamine (DFO) producing thiourea linkages with the amino group of surface-exposed lysine residues. Successful DFO-conjugation was confirmed by SDS-PAGE analysis (FIG. 7). Size exclusion chromatography analysis of DFO-conjugated antibody fragments revealed a single peak with slightly earlier elution time (compared with the unconjugated fragments), corresponding to a molecular weight shift confirming the addition of the chelator.

DFO-conjugated A2SM antibody fragment was radiolabeled by chelation of ⁸⁹Zr with similar results for labeling efficiencies (around 90%), resulting in specific activities of 4.5 μCi/μg and yielding radiochemical purities of >95%.

### Example 5: 89Zr-A2SM immunoPET in a Syngeneic Prostate Cancer Model (RM9-hPSCA) in Human PSCA Knock-In Mice (hPSCAKI)

Unless otherwise specified below, the materials and methods are the same as those disclosed above in Example 1.

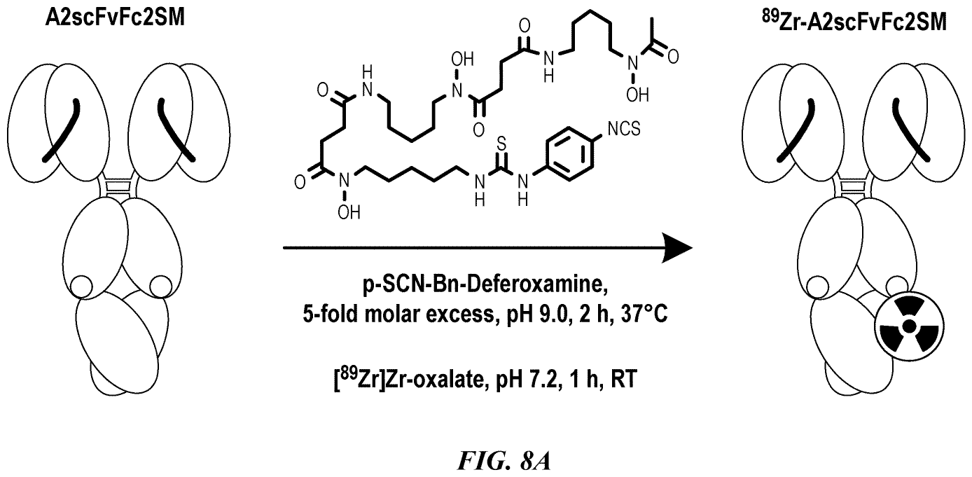

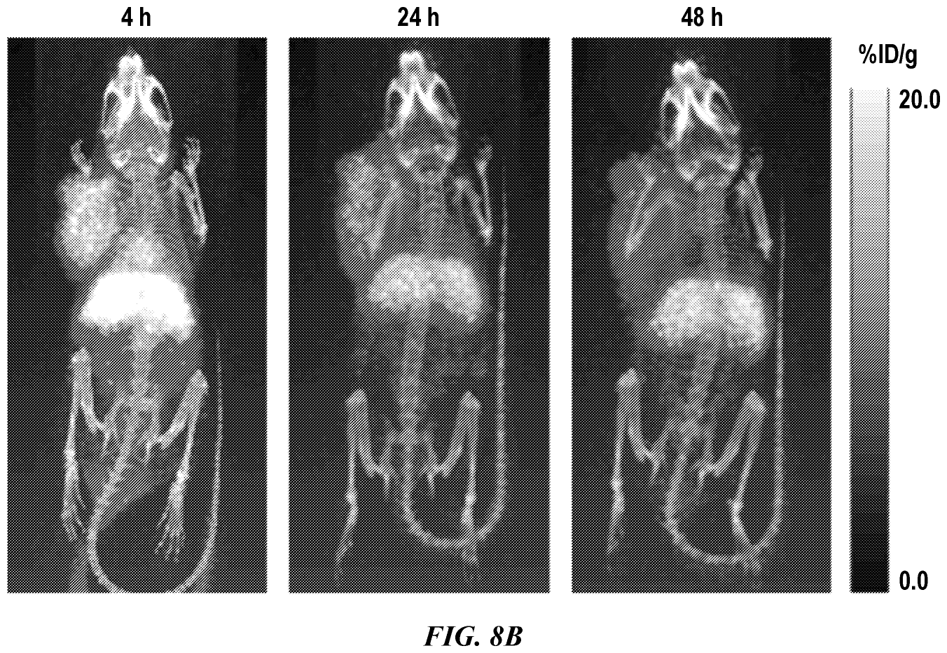

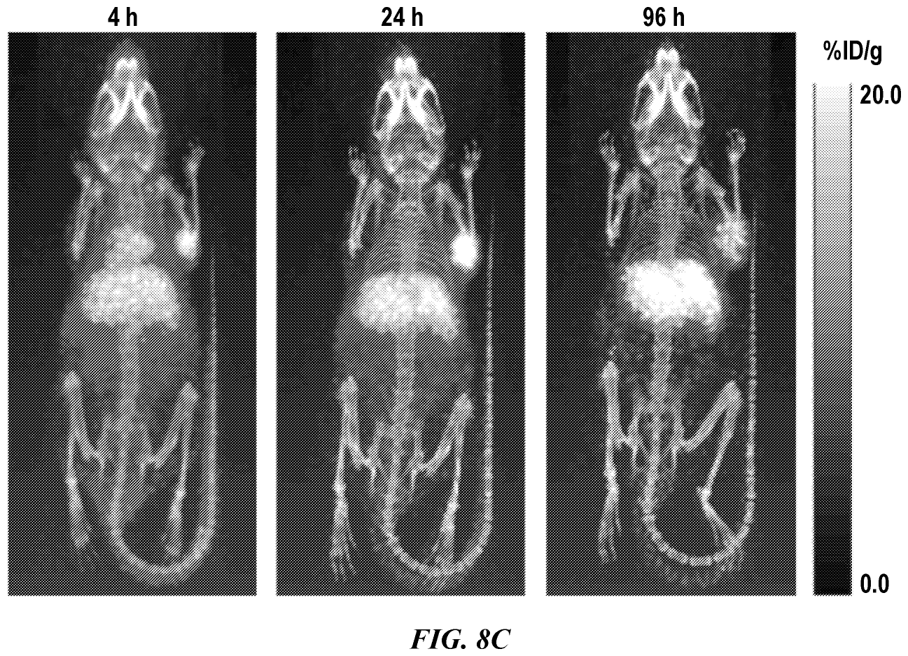

Male hPSCA KI mice bearing RM9 or RM9-hPSCA subcutaneous tumors were injected with ⁸⁹Zr-A2SM (45 μCi/1.7 MBq/10 μg) via the tail vein. ImmunoPET/CT scans were acquired at specific time points post injection (4, 24, and 48 h for RM9 and 4, 24, and 96 h for RM9-hPSCA). Representative images are shown in FIG. 8. Antigen-specific uptake was observed in mice bearing PSCA positive tumors (RM9-hPSCA) as early as 4 h p.i. and peaked around 24 h p.i. In mice bearing antigen negative RM9 tumors some signal in the tumors (from blood pool activity or EPR effects) was observed at 4 h p.i. and continuously decreased. Blood pool activity can be seen in the heart at early time points and rapid clearance of the tracer to the liver is seen throughout the imaging study. After the last scan, mice were euthanized and tissues harvested for ex vivo biodistribution confirming specific uptake in PSCA-positive tumors (18.0±0.2% ID/g at 96 h p.i., n=3) compared to PSCA-negative tumors (8.2±0.95% ID/g at 48 h p.i., n=5).

Anti-PSCA A2 scFv-Fc2 antibody fragments with one mutation (single mutant, A2SM) or with two mutations and genetically aglycosylated (A2aG) were produced by mammalian expression. Purified proteins were analyzed by SDS-PAGE and size exclusion chromatography (SEC). Specific binding to recombinant antigen (ELISA) and PSCA-positive cells (flow cytometry) was compared to parental variants. A2SM was conjugated with SCN-DFO and subsequently radiolabeled with ⁸⁹Zr. ⁸⁹Zr-Df-A2SM (10 μg/1.67 MBq) was injected i.v. into hPSCA KI mice (C57BL6/JxhPSCA) bearing syngeneic prostate cancer tumors (RM9 or RM9-hPSCA) and PET/CT scans (Molecubes) were acquired at 4, 24, 44, 96 h p.i. After the last scan, ex vivo biodistribution were conducted and % ID/g calculated based on decay-corrected standards.

Two novel anti-PSCA scFv-Fc variants (A2SM, A2aG) were designed, produced, and purified (FIG. 1). SDS-PAGE and SEC analysis confirmed purity and dimeric self-assembly. Saturation binding studies showed both A2SM and A2aG retained antigen specific binding and affinity to both recombinant hPSCA and PSCA-expressing cells (K_{d}: 3.3±0.5 nM and 3.1±0.5 nM, respectively). A2SM was successfully radiolabeled with ⁸⁹Zr (efficiency: >90%, purity >99%, n=2) resulting in a specific activity of ~0.17 MBq/μg (FIG. 2). ImmunoPET showed specific uptake in RM9-hPSCA s.c. tumors (18.0±0.2% ID/g at 96 h p.i., n=3) and was significantly higher than in RM9 tumors (8.2±1.0% ID/g at 48 h p.i., n=5). The ⁸⁹Zr-A2SM exhibited slower blood clearance compared with ⁸⁹Zr-A2DM, leading to higher total tumor uptake but lower tumor-to-blood ratio (7.2±1.2 vs 25.8±2.5 at 96 h p.i., n≥3). Therefore, the novel A2SM could deliver a higher cytotoxic dose to the tumor while the liver remains the dose-limiting organ.

Surrogate ⁸⁹Zr-immunoPET successfully profiled the in vivo tumor targeting, biodistribution and clearance of the novel PK-modulated anti-PSCA A2SM in hPSCA KI mice. ⁸⁹Zr-A2SM with an intermediate blood half-live reached higher uptake in PSCA-expressing tumors and higher tumor-to-liver ratios than ⁸⁹Zr-A2DM. Dosimetry estimations will help predict if A2SM will translate to a higher TI for RIT of PSCA-expressing cancers.

### Example 6: Development of a Near-Infrared Fluorescent (NIRF)-Conjugated Anti-PSCA Antibody Fragment (A2scFv-Fc2 DM-IRDye800CW) for Intraoperative Guidance

The mainstay of treatment for localized prostate cancer is surgery and the outcome depends on complete resection, that is negative tumor margins and identification and resection of metastatic disease to pelvic lymph nodes. The prostate stem cell antigen (PSCA) is highly expressed in both localized and metastatic prostate cancer and can be targeted using engineered antibody fragments with high specificity and optimized pharmacokinetics. The PK-modulated scFv-Fc antibody fragment A2scFv-Fc2 DM (A2DM), with rapid blood clearance and elimination by the liver, can be combined with near-infrared fluorescent dyes and provide high-contrast optical imaging for visualization of prostate cancer.

### Methods

A2scFv-Fc2 DM Conjugation with IRDye800CW

Anti-PSCA A2DM was randomly conjugated to amine-reactive NHS-IRDye800CW (molar ratio dye:protein 5-30) resulting in A2DM-NHS800 or site-specifically conjugated (after mild reduction with 5-fold molar excess of TCEP, 60 min, RT) using thiol-reactive maleimide-IRDye800CW (molar ratio dye:protein 5-30) and resulting in A2DM-mal800.

Dye-to-protein ratio (DPR) was determined after the removal of excess dye via size exclusion spin column (Micro Bio-Spin, BioRAD) by spectrophotometer (NanoDrop One). The purity and integrity of the fluorescent-labeled proteins were analyzed by SDS-PAGE and Size exclusion chromatography.

### Antigen Binding Affinity (ELISA)

Recombinant human PSCA-murine Fc fusion protein (hPSCA-mFc) was immobilized (1 μg/mL) in ELISA plates overnight at 4° C. Wells were blocked (PBS 1% BSA) and serial dilution of fluorescent-labeled antibodies (0.2 μM-500 nM) were incubated for 1 hour at room temperature. After washing (3 times PBS, 1% Tween-20), bound antibody was detected using goat anti-human IgG H&L-AP (Abcam, ab6859) followed by 1-Step PNPP substrate solution (ThermoFisher, 37621). The reaction was stopped by adding 2 M NaOH and absorbance was read at 405 nm.

### Binding to Antigen-Expressing Cells (Flow Cytometry)

Murine prostate cancer cells (RM9 and RM9-hPSCA, 1×10⁶ cells/100 μL PBS, 1% FBS) were incubated with A2DM, A2DM-NHS800 or A2DM-mal800 (1 ug/mL) for 1 h at 4° C. After washing, bound antibody was detected using a goat anti-human IgG-AlexaFluor 647 secondary antibody (1:1000, Invitrogen), and cells were analyzed on a BD LSRFortessa X-20 Cell Analyzer.

### ⁸⁹Zr-immunoPET Imaging of A2DM-NHS800 and A2DM-Mal800 in Human PSCA KI Mice Bearing RM9/RM9-hPSCA Tumors

The lead candidates for both random (A2DM-NHS800) and site-specific (A2DM-mal800) conjugations were conjugated with p-SCN-Bn-Deferoxamine (DFO) and subsequently radiolabeled with ⁸⁹Zr. Radiolabeling efficiency, specific activity and radiochemical purity were assessed using instant-thin layer chromatography (ITLC).

Human PSCA knock-in C57BL/6J mice (hPSCA KI) bearing subcutaneous RM9-hPSCA tumors on the right shoulder and antigen-negative RM9 tumors on the left side will be injected with ⁸⁹Zr-labeled A2DM, A2DM-NHS800, or A2DM-mal800 (50 μCi/10 μg) (groups of n≥5). PET/CT imaging was performed at 4, 24, and 48 h post-injection. After the last scan, mice were euthanized for ex vivo biodistribution, tissues were weighed and gamma-counted to calculate the % ID/g.

### Optical Near-Infrared Fluorescence Imaging of A2DM-NHS800 and A2DM-Mal800

Tumor-bearing (RM9 and RM9-hPSCA s.c.) hPSCA KI mice were injected i.v. with 10 μg of A2DM-NHS800 or A2DM-mal800. At 24 or 48 h p.i. mice were euthanized and imaged in the Pearl Trilogy NIRF small animal imaging system (LI-COR). Images were taken with the skin removed to simulate a surgical setting. Tumors and tissues of interest were dissected and imaged ex vivo for direct comparison.

### Results

### Random and Site-Specific Conjugation of A2DM and IRDye800CW

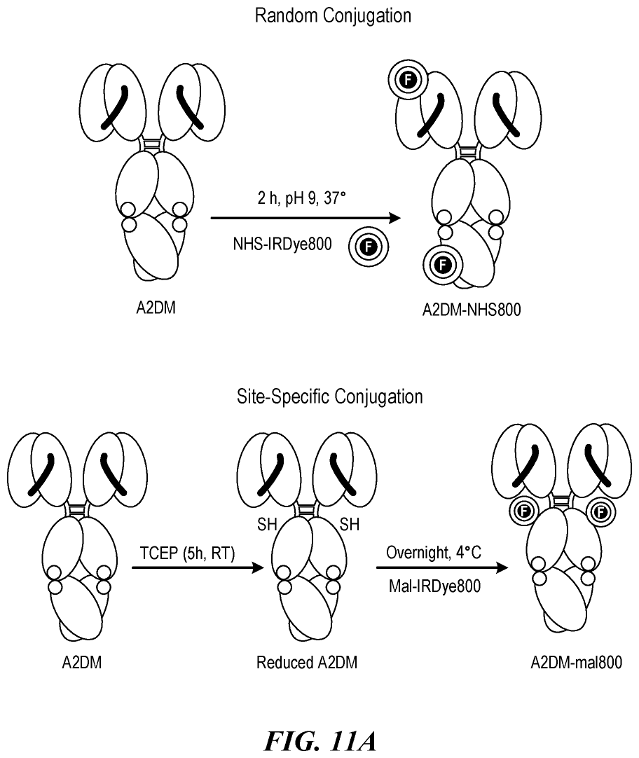

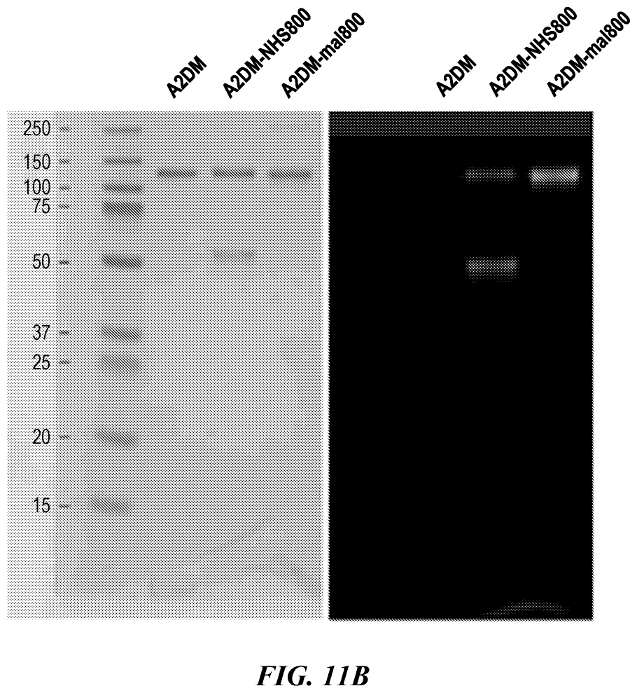

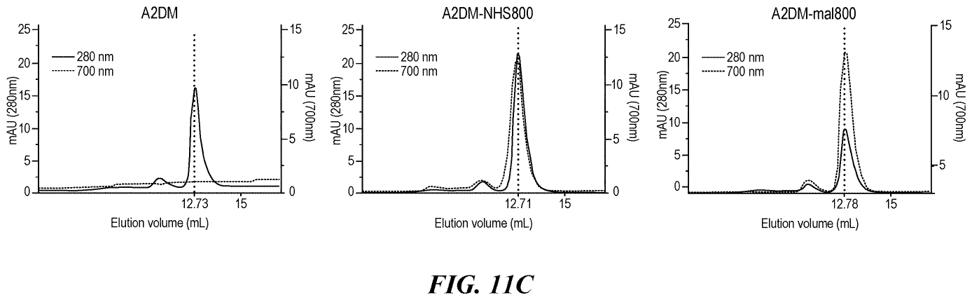

The A2DM was successfully conjugated with IRDye800CW using both random and site-specific chemistry, as shown in FIG. 11A. The resulting dye-to-protein ratios (DPR) ranged from 0.5-1.3. SDS-PAGE analysis confirmed the purity of the conjugated protein and fluorescent signal coinciding with the protein bands (FIG. 11B). SEC results indicate that A2DM retained its dimeric conformation and concurrent elution of the fluorophore confirmed successful conjugation (FIG. 11C).

### Antigen Binding Specificity and Affinity

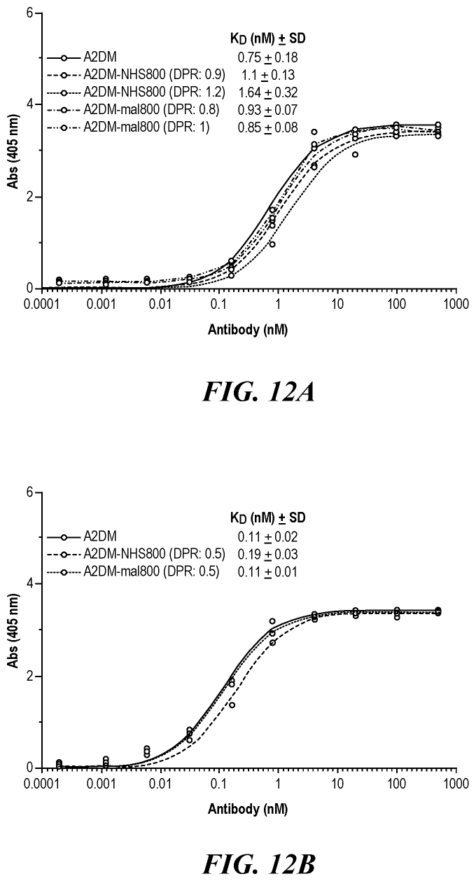

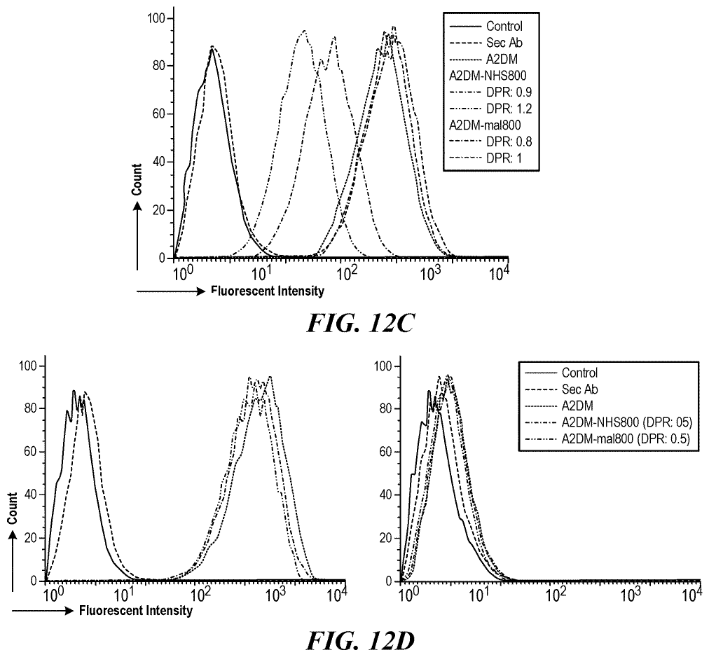

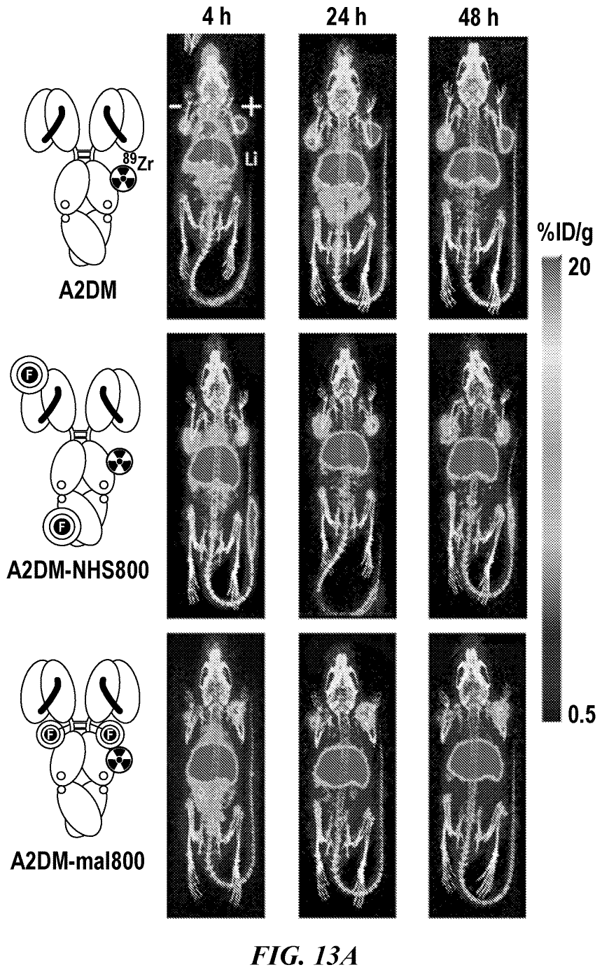

A2DM-NHS800 and A2DM-mal800 at dye-to-protein ratios ranging from 0.5-1.3 were compared to unconjugated A2DM to assess the impact of NIRF dye addition on the antibody fragments' capability to bind immobilized antigen (FIG. 12A). The conjugated antibody fragments showed binding with comparable sub-nanomolar EC50 values at a lower DPR of 0.5 (FIG. 12B). The randomly conjugated A2DM-NHS800 showed slightly lower affinity at DPR >1.

Binding to cell surface antigen was tested by flow cytometry using the murine prostate cancer cells RM9 transduced with human PSCA (RM9-hPSCA) or wild-type cells as antigen-negative control (FIG. 12C). The site-specifically conjugated A2DM-mal800 showed antigen-specific binding to RM9-hPSCA cells that was comparable to that of unconjugated A2DM and was not affected by the addition of IRDye800 at DPR up to 1.0. The randomly conjugated A2DM-NHS800 showed reduced binding at DPR 0.9 and more so at DPR 1.2. Therefore, the DPR of 0.5 was chosen for both conjugation methods for all subsequent experiments, and similar binding to RM9-hPSCA was confirmed in comparison to A2DM (FIG. 12D). The specificity of binding was tested using RM9 cells and no binding was detected.

### ⁸⁹Zr-immunoPET Profiles In Vivo Tumor Targeting, Biodistribution and Clearance of A2DM-IRDye800CW

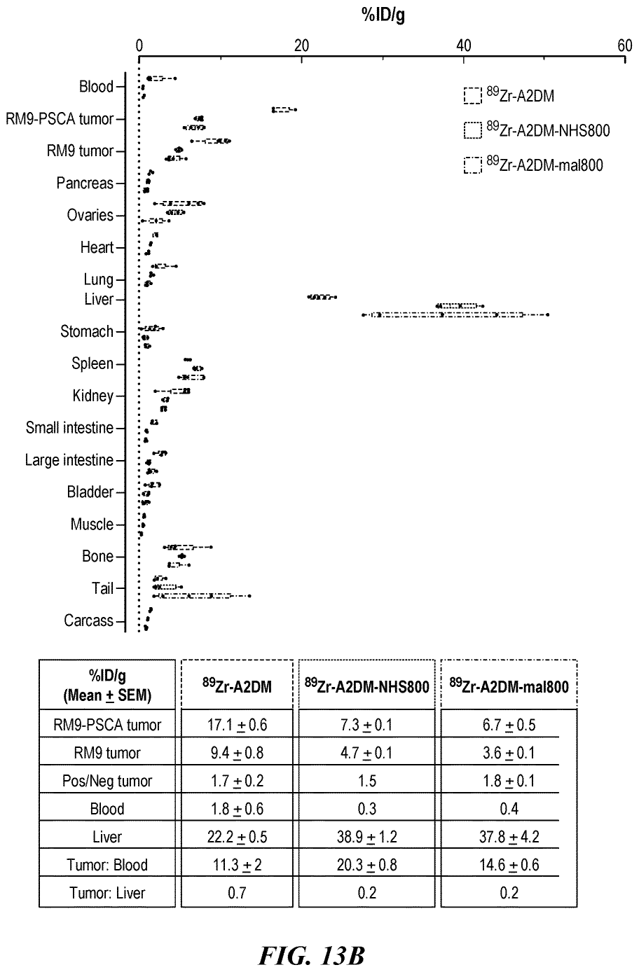

To examine the antigen-specific tumor targeting, the pharmacokinetic profile, and the biodistribution of A2DM, A2DM-NHS800, and A2DM-mal800 we radiolabeled them by DFO-chelation of ⁸⁹Zr after random conjugation to lysines (p-SCN-Bn-DFO), as shown in FIG. 13A. Radiolabeling resulted in labeling efficiencies >80% with specific activities around 5 μCi/μg and radiochemical purities after size exclusion chromatography were >95%. Human PSCA KI mice (hPSCA KI) with bilateral RM9-hPSCA (right shoulder) and RM9 (left shoulder) s.c. tumors were injected i.v. with 10 μg protein (50 Ci/1.85 MBq) and serial immunoPET/CT imaging (4, 24, 48 h p.i.) showed antigen-specific accumulation of signal that was higher in the RM9-hPSCA tumors compared to the antigen-negative RM9 tumors, as shown in FIG. 13B. All antibody fragments showed early accumulation of signal in the liver and both NIRF-conjugated A2DM variants cleared more rapidly than the ⁸⁹Zr-A2DM. The faster blood clearance of ⁸⁹Zr-A2DM-NHS-800 and ⁸⁹Zr-A2DM-mal800 resulted in lower total uptake in the RM9-hPSCA tumors (7.3±0.1 and 6.7±0.5% ID/g, respectively) compared with ⁸⁹Zr-A2DM (17.1±0.6% ID/g), however, the tumor-to-blood ratios were higher (20.3±0.8 and 14.6±0.6 compared to 11.3±2.0), which should translate into reaching good imaging contrast at earlier time points.

Optical Near-Infrared Fluorescence Imaging (A2DM-NHS800 and A2DM-Mal800) of hPSCA KI Mice Bearing PSCA-Expressing RM9-hPSCA Tumors

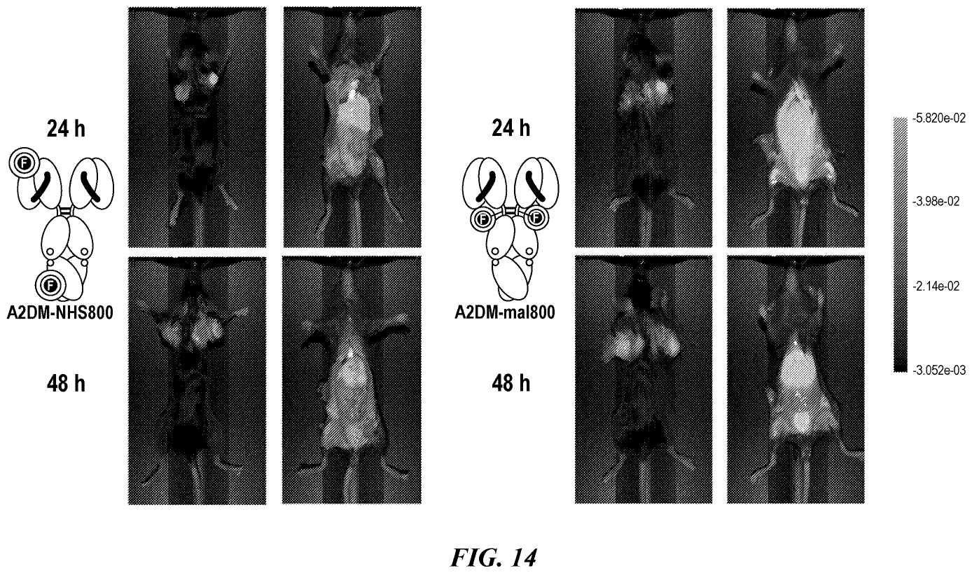

Mice were injected with 10 μg protein (A2DM-NHS800 or A2DM-mal800) via the tail vein and groups of mice were euthanized at 24 or 48 h p.i. for optical imaging using the Pearl Trilogy Imaging system. Mice were imaged with the skin removed to simulate a surgical situation (FIG. 14). The fluorescent signal accumulated in the RM9-hPSCA tumors on the right shoulder with high contrast to the surrounding muscle tissue.

A minimal fluorescent signal was observed in the negative RM9 tumors on the left shoulder. Non-specific signal was seen in the liver, confirming the biodistribution seen in the immunoPET studies. At 48 h p.i. the fluorescent signal in the RM9-hPSCA tumors had decreased slightly, however, the contrast to surrounding tissues was higher. The site-specifically conjugated A2DM-mal800 showed minimally higher non-specific signal in the intestines, kidneys, and bladder.

In summary, the optical imaging showed excellent antigen-specific tumor delineation with a possible surgical window of at least from 24-48 h p.i., resulting in high contrast to surrounding healthy tissue.

### ENUMERATED EMBODIMENTS

Clause 1. A genetically engineered anti-prostate stem cell antigen (PSCA) scFv-Fc fusion protein comprising two peptides which form a homodimer, wherein each peptide comprises variable domains VH and VL of an anti-PSCA antibody, and a truncated hinge and fragment crystallizable (Fc) region, wherein the variable domains are arranged in the order of VH-VL, and wherein the FcRn binding region of the Fc region comprises one or more first point mutations.
Clause 2. The fusion protein of clause 1, wherein the variable domains VH and VL are connected by a glycine-rich linker
Clause 3. The fusion protein of clause 2, wherein the linker is about 10-25 amino acids.
Clause 4. The fusion protein of clause 2 or claim 3, wherein the linker has a sequence of ((G4S) 2-GGSAQ) (SEQ ID NO: 1).
Clause 5. The fusion protein of any one of clauses 1-4, wherein the one or more first point mutations reduces hematological toxicity.
Clause 6. The fusion protein of any one of clauses 1-5, wherein the one or more first point mutations is selected from (i) H310A (ii) H435Q, or (iii) both H310A and H435Q.
Clause 7. The fusion protein of any one of clauses 1-6, wherein the truncated hinge and Fc region comprises a second point mutation that is N297Q, and wherein the second point mutation removes an N-glycosylation site.
Clause 8. The fusion protein of any one of clauses 1-7, wherein the truncated hinge and Fc region comprises a third point mutation that is K322A.
Clause 9. The fusion protein of any one of clauses 1-8, wherein the truncated hinge and Fc region is derived from human IgG2.
Clause 10. The fusion protein of any one of clauses 1-9, wherein each peptide of the scFv-Fc fusion protein has an amino acid sequence at least 80%, at least 85%, at least 90%, at least 95%, at least 98%, or at least 99% identical to SEQ ID NO: 2, SEQ ID NO: 3, or SEQ ID NO: 5.
Clause 11. The fusion protein of any one of clauses 1-10, wherein the scFv-Fc fusion protein is conjugated to an effector moiety.
Clause 12. The fusion protein of clause 11, wherein the effector moiety includes a labeling moiety and/or a therapeutic moiety.
Clause 13. The fusion protein of clause 12, wherein the labeling moiety comprises one or more radioactive labels or fluorescent labels. 14 The fusion protein of claim 12, wherein the therapeutic moiety includes a cytotoxic agent, an anti-tumor drug, a toxin, a radioactive agent, a cytokine, a second protein, an antibody, a radionuclide, or an enzyme.
Clause 15. A pharmaceutical composition comprising an effective amount of one or more scFv-Fc fusion proteins, or scFv-Fc fusion protein and effector moiety conjugates of any one of clauses 1-14.
Clause 16. The pharmaceutical composition of clause 15, further comprising one or more pharmaceutically acceptable carriers, excipients, and/or stabilizers.
Clause 17. The pharmaceutical composition of clause 15 or claim 16, further comprising one or more additional therapeutic agents.
Clause 18. The pharmaceutical composition of clause 17, wherein the one or more additional therapeutic agents are conjugated to the scFv-Fc fusion protein.
Clause 19. The pharmaceutical composition of clause 17 or claim 18, wherein the additional therapeutic agents include chemotherapeutic agents, cytotoxic agents, cytokines, growth inhibitory agents, and anti-hormonal agents.
Clause 20. The pharmaceutical composition of any one of clauses 15-19, wherein the pharmaceutical composition is formulated suitable for intravenous, intramuscular, intraperitoneal, intracerobrospinal, subcutaneous, intraarticular, intrasynovial, intrathecal, oral, topical, or inhalation administration.
Clause 21. A method of treating or preventing a cancer expressing PSCA comprising administering to a subject an effective amount of one or more scFv-Fc fusion proteins, scFv-Fc fusion protein and effector moiety conjugates, or pharmaceutical compositions of any one of clauses 1-20.
Clause 22. The method of clause 21, further comprising administering to the subject one or more additional therapeutic agents.
Clause 23. The method of clause 22, wherein the additional therapeutic agents include chemotherapeutic agents, radiotherapy, cytotoxic agents, cytokines, growth inhibitory agents, and anti-hormonal agents.
Clause 24. A method of detecting cancer expressing PSCA in a subject, comprising: administering one or more scFv-Fc fusion proteins of any one of clauses 1-14 to a subject; measuring the level of the scFv-Fc fusion proteins in the subject; and comparing the level of the scFv-Fc fusion proteins with that of a healthy subject or with an average level of a healthy population, wherein an elevated level of the scFv-Fc fusion proteins in the subject indicating the presence of cancer.
Clause 25. The method of clause 24, wherein the scFv-Fc fusion protein is conjugated with a labeling moiety.
Clause 26. The method of clause 25, wherein the labeling moiety comprises one or more radioactive isotopes such as ³²P, ^{99m}Tc, ¹¹¹In, ¹⁸F, ⁶⁴Cu, and ⁸⁹Zr, fluorescent dyes, electron-dense reagents, enzymes, biotin, digoxigenin, or haptens and proteins which can be made detectable.
Clause 27. A method of determining the prognosis of treating a cancer expressing PSCA in a subject, comprising: administering one or more scFv-Fc fusion proteins of any one of clauses 1-15 to a subject; measuring the level of the scFv-Fc fusion proteins in the subject; and comparing the level of the scFv-Fc fusion proteins before and after the subject receives a cancer therapy, wherein a decreased level of the scFv-Fc fusion proteins in the subject after receiving the cancer therapy indicating that the cancer therapy is effective.
Clause 28. The method of clause 27, wherein the scFv-Fc fusion protein is conjugated with a labeling moiety.
Clause 29. The method of clause 28, wherein the labeling moiety comprises one or more radioactive isotopes such as ³²P, ^{99m}Tc, ¹¹¹In, ¹⁸F, ⁶⁴Cu, and ⁸⁹Zr, fluorescent dyes, electron-dense reagents, enzymes, biotin, digoxigenin, or haptens and proteins which can be made detectable.
Clause 30. A method of imaging a cancer expressing PSCA in a subject, comprising: administering one or more scFv-Fc fusion proteins of any one of clauses 1-15 to a subject; and imaging the subject to determine the location and size of the tumor.
Clause 31. The method of clause 30, wherein the scFv-Fc fusion protein is conjugated with a labeling moiety.
Clause 32. The method of clause 31, wherein the labeling moiety comprises one or more radioactive isotopes such as ³²P, ^{99m}Tc, ¹¹¹In, ¹⁸F, ⁶⁴Cu, and ⁸⁹Zr, fluorescent dyes, electron-dense reagents, enzymes, biotin, digoxigenin, or haptens and proteins which can be made detectable.
Clause 33. The method of any one of clauses 21-32, wherein the cancer expressing PSCA includes prostate cancer, pancreatic cancer, and bladder cancer.
Clause 34. The method of clause 31, wherein the labeling moiety is IRDye800CW.

1. A genetically engineered anti-prostate stem cell antigen (PSCA) scFv-Fc fusion protein comprising two peptides which form a homodimer, wherein each peptide comprises variable domains VH and VL of an anti-PSCA antibody, and a truncated hinge and fragment crystallizable (Fc) region, wherein the variable domains are arranged in the order of VH-VL, and wherein the FcRn binding region of the Fc region comprises one or more first point mutations.

2. The fusion protein of claim 1, wherein the variable domains VH and VL are connected by a glycine-rich linker

3. The fusion protein of claim 2, wherein the linker is about 10-25 amino acids.

4. The fusion protein of claim 2 or claim 3, wherein the linker has a sequence of ((G4S) 2-GGSAQ) (SEQ ID NO: 1).

5. The fusion protein of any one of claims 1-4, wherein the one or more first point mutations reduces hematological toxicity.

6. The fusion protein of any one of claims 1-5, wherein the one or more first point mutations is selected from (i) H310A (ii) H435Q, or (iii) both H310A and H435Q.

7. The fusion protein of any one of claims 1-6, wherein the truncated hinge and Fc region comprises a second point mutation that is N297Q, and wherein the second point mutation removes an N-glycosylation site.

8. The fusion protein of any one of claims 1-7, wherein the truncated hinge and Fc region comprises a third point mutation that is K322A.

9. The fusion protein of any one of claims 1-8, wherein the truncated hinge and Fc region is derived from human IgG2.

10. The fusion protein of any one of claims 1-9, wherein each peptide of the scFv-Fc fusion protein has an amino acid sequence at least 80%, at least 85%, at least 90%, at least 95%, at least 98%, or at least 99% identical to SEQ ID NO: 2, SEQ ID NO: 3, or SEQ ID NO: 5.

11. The fusion protein of any one of claims 1-10, wherein the scFv-Fc fusion protein is conjugated to an effector moiety.

12. The fusion protein of claim 11, wherein the effector moiety includes a labeling moiety and/or a therapeutic moiety.

13. The fusion protein of claim 12, wherein the labeling moiety comprises one or more radioactive labels or fluorescent labels.

14. The fusion protein of claim 12, wherein the therapeutic moiety includes a cytotoxic agent, an anti-tumor drug, a toxin, a radioactive agent, a cytokine, a second protein, an antibody, a radionuclide, or an enzyme.

15. A pharmaceutical composition comprising an effective amount of one or more scFv-Fc fusion proteins, or scFv-Fc fusion protein and effector moiety conjugates of any one of claims 1-14.

16. The pharmaceutical composition of claim 15, further comprising one or more pharmaceutically acceptable carriers, excipients, and/or stabilizers.

17. The pharmaceutical composition of claim 15 or claim 16, further comprising one or more additional therapeutic agents.

18. The pharmaceutical composition of claim 17, wherein the one or more additional therapeutic agents are conjugated to the scFv-Fc fusion protein.

19. The pharmaceutical composition of claim 17 or claim 18, wherein the additional therapeutic agents include chemotherapeutic agents, cytotoxic agents, cytokines, growth inhibitory agents, and anti-hormonal agents.

20. The pharmaceutical composition of any one of claims 15-19, wherein the pharmaceutical composition is formulated suitable for intravenous, intramuscular, intraperitoneal, intracerobrospinal, subcutaneous, intraarticular, intrasynovial, intrathecal, oral, topical, or inhalation administration.

21. A method of treating or preventing a cancer expressing PSCA comprising administering to a subject an effective amount of one or more scFv-Fc fusion proteins, scFv-Fc fusion protein and effector moiety conjugates, or pharmaceutical compositions of any one of claims 1-20.

22. The method of claim 21, further comprising administering to the subject one or more additional therapeutic agents.

23. The method of claim 22, wherein the additional therapeutic agents include chemotherapeutic agents, radiotherapy, cytotoxic agents, cytokines, growth inhibitory agents, and anti-hormonal agents.

24. A method of detecting cancer expressing PSCA in a subject, comprising:
administering one or more scFv-Fc fusion proteins of any one of claims 1-14 to a subject;
measuring the level of the scFv-Fc fusion proteins in the subject; and
comparing the level of the scFv-Fc fusion proteins with that of a healthy subject or with an average level of a healthy population,
wherein an elevated level of the scFv-Fc fusion proteins in the subject indicating the presence of cancer.

25. The method of claim 24, wherein the scFv-Fc fusion protein is conjugated with a labeling moiety.

26. The method of claim 25, wherein the labeling moiety comprises one or more radioactive isotopes such as ³²P, ^{99m}Tc, ¹¹¹In, ¹⁸F, ⁶⁴Cu, and ⁸⁹Zr, fluorescent dyes, electron-dense reagents, enzymes, biotin, digoxigenin, or haptens and proteins which can be made detectable.

27. A method of determining the prognosis of treating a cancer expressing PSCA in a subject, comprising:
administering one or more scFv-Fc fusion proteins of any one of claims 1-15 to a subject;
measuring the level of the scFv-Fc fusion proteins in the subject; and
comparing the level of the scFv-Fc fusion proteins before and after the subject receives a cancer therapy,
wherein a decreased level of the scFv-Fc fusion proteins in the subject after receiving the cancer therapy indicating that the cancer therapy is effective.

28. The method of claim 27, wherein the scFv-Fc fusion protein is conjugated with a labeling moiety.

29. The method of claim 28, wherein the labeling moiety comprises one or more radioactive isotopes such as ³²P, ^{99m}Tc, ¹¹¹In, ¹⁸F, ⁶⁴Cu, and ⁸⁹Zr, fluorescent dyes, electron-dense reagents, enzymes, biotin, digoxigenin, or haptens and proteins which can be made detectable.

30. A method of imaging a cancer expressing PSCA in a subject, comprising:
administering one or more scFv-Fc fusion proteins of any one of claims 1-15 to a subject; and
imaging the subject to determine the location and size of the tumor.

31. The method of claim 30, wherein the scFv-Fc fusion protein is conjugated with a labeling moiety.

32. The method of claim 31, wherein the labeling moiety comprises one or more radioactive isotopes such as ³²P, ^{99m}Tc, ¹¹¹In, ¹⁸F, ⁶⁴Cu, and ⁸⁹Zr, fluorescent dyes, electron-dense reagents, enzymes, biotin, digoxigenin, or haptens and proteins which can be made detectable.

33. The method of any one of claims 21-32, wherein the cancer expressing PSCA includes prostate cancer, pancreatic cancer, and bladder cancer.

34. The method of claim 31, wherein the labeling moiety is IRDye800CW.


SEQUENCE LISTING (7 sequences)

SEQ ID NO: 1 - protein, 5 aa, synthetic construct
GGSAQ

SEQ ID NO: 2 - protein, 456 aa, synthetic construct
EVQLVESGGGLVQPGGSLRLSCAASGFNIKDYYIHWVRQAPGKGLEWVAWIDPEYGDSEFVPKFQGRATMSADTSKNTAYLQMNSLRAEDTAVYYCKTGGFWGRGTLVTVSSGGGGSGGGGSGGSAQDIQLTQSPSSLSASVGDRVTITCSASSSVRFIHWYQQKPGKAPKRLIYDTSKLASGVPSRFSGSGSGTDFTLTISSLQPEDFATYYCQQWGSSPFTFGQGTKVEIKVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - DNA, 1368 bp, synthetic construct
  source 1..1368: mol_type = other DNA
gaggtgcagcttgttgaatctggcggaggactggttcagcctggcggatctctgagactgtcttgtgccgccagcggcttcaacatcaaggactactacatccactgggtccgacaggcccctggcaaaggacttgaatgggtcgcctggatcgaccccgagtacggcgattctgagttcgtgcctaagttccagggcagagccacaatgagcgccgacacctctaagaacaccgcctacctgcagatgaacagcctgagagccgaggacaccgccgtgtactattgcaagacaggcggcttttggggcagaggcacactggtcacagttagctctggcggcggaggaagcggaggcggaggatcaggcggatctgctcaggatatccagctgacacagagcccctctagcctgtctgctagcgtgggcgacagagtgaccatcacatgtagcgcctctagcagcgtgcggtttatccactggtatcagcagaagcccggcaaggcccctaagagactgatctacgacaccagcaagctggcctctggcgtgccatctagattcagcggctctggctctggcaccgacttcaccctgacaatcagtagcctgcagcctgaggacttcgccacctactactgtcagcagtggggcagcagccctttcacatttggccagggcaccaaggtggaaatcaaggtcgagtgccctccttgtcctgctcctcctgttgctggcccttccgtgttcctgtttcctccaaagcctaaggacaccctgatgatcagcaggacccctgaagtgacctgcgtggtggtggatgtgtctcacgaggatcctgaggtgcagttcaattggtacgtggacggcatggaagtgcacaacgccaagaccaagcctagagaggaacagttcaacagcaccttcagagtggtgtccgtgctgaccgtggtggctcaggattggctgaacggcaaagagtacaagtgcgccgtgtccaacaagggcctgcctgctcctatcgaaaagaccatcagcaagacaaagggacagccccgcgagcctcaggtgtacacacttcctccaagcagagaagagatgaccaagaaccaggtgtccctgacctgcctggtcaagggcttctacccttccgatatcgccgtggaatgggagagcaatggccagccagagaacaactacaagaccacacctcctatgctggacagcgacggctcattcttcctgtactccaagctgacagtggacaagagcagatggcagcagggcaacgtgttcagctgcagcgtgatgcacgaggccctgcacaaccactacacccagaagtccctgagcctgtctcctggcaaa

SEQ ID NO: 4 - protein, 456 aa, synthetic construct
EVQLVESGGGLVQPGGSLRLSCAASGFNIKDYYIHWVRQAPGKGLEWVAWIDPEYGDSEFVPKFQGRATMSADTSKNTAYLQMNSLRAEDTAVYYCKTGGFWGRGTLVTVSSGGGGSGGGGSGGSAQDIQLTQSPSSLSASVGDRVTITCSASSSVRFIHWYQQKPGKAPKRLIYDTSKLASGVPSRFSGSGSGTDFTLTISSLQPEDFATYYCQQWGSSPFTFGQGTKVEIKVECPPCPAPPVAGPSVFLFPPKPKDTLMISRTPEVTCVVVDVSHEDPEVQFNWYVDGMEVHNAKTKPREEQFNSTFRVVSVLTVVAQDWLNGKEYKCAVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 5 - DNA, 1368 bp, synthetic construct
  source 1..1368: mol_type = other DNA
gaggtgcagcttgttgaatctggcggaggactggttcagcctggcggatctctgagactgtcttgtgccgccagcggcttcaacatcaaggactactacatccactgggtccgacaggcccctggcaaaggacttgaatgggtcgcctggatcgaccccgagtacggcgattctgagttcgtgcctaagttccagggcagagccacaatgagcgccgacacctctaagaacaccgcctacctgcagatgaacagcctgagagccgaggacaccgccgtgtactattgcaagacaggcggcttttggggcagaggcacactggtcacagttagctctggcggcggaggaagcggaggcggaggatcaggcggatctgctcaggatatccagctgacacagagcccctctagcctgtctgctagcgtgggcgacagagtgaccatcacatgtagcgcctctagcagcgtgcggtttatccactggtatcagcagaagcccggcaaggcccctaagagactgatctacgacaccagcaagctggcctctggcgtgccatctagattcagcggctctggctctggcaccgacttcaccctgacaatcagtagcctgcagcctgaggacttcgccacctactactgtcagcagtggggcagcagccctttcacatttggccagggcaccaaggtggaaatcaaggtcgagtgccctccttgtcctgctcctcctgttgctggcccttccgtgttcctgtttcctccaaagcctaaggacaccctgatgatcagcaggacccctgaagtgacctgcgtggtggtggatgtgtctcacgaggatcctgaggtgcagttcaattggtacgtggacggcatggaagtgcacaacgccaagaccaagcctagagaggaacagttccaaagcaccttcagagtggtgtccgtgctgaccgtggtggctcaggattggctgaacggcaaagagtacaagtgcgccgtgtccaacaagggcctgcctgctcctatcgaaaagaccatcagcaagacaaagggacagccccgcgagcctcaggtgtacacacttcctccaagcagagaagagatgaccaagaaccaggtgtccctgacctgcctggtcaagggcttctacccttccgatatcgccgtggaatgggagagcaatggccagccagagaacaactacaagaccacacctcctatgctggacagcgacggctcattcttcctgtactccaagctgacagtggacaagagcagatggcagcagggcaacgtgttcagctgcagcgtgatgcacgaggccctgcacaaccagtacacccagaagtccctgagcctgtctcctggcaaa

SEQ ID NO: 6 - protein, 456 aa, synthetic construct
EVQLVESGGGLVQPGGSLRLSCAASGFNIKDYYIHWVRQAPGKGLEWVAWIDPEYGDSEFVPKFQGRATMSADTSKNTAYLQMNSLRAEDTAVYYCKTGGFWGRGTLVTVSSGGGGSGGGGSGGSAQDIQLTQSPSSLSASVGDRVTITCSASSSVRFIHWYQQKPGKAPKRLIYDTSKLASGVPSRFSGSGSGTDFTLTISSLQPEDFATYYCQQWGSSPFTFGQGTKVEIKVECPPCPAPPVAGPSVFLFPPKPKDTLMISRTPEVTCVVVDVSHEDPEVQFNWYVDGMEVHNAKTKPREEQFQSTFRVVSVLTVVAQDWLNGKEYKCAVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNQYTQKSLSLSPGK

SEQ ID NO: 7 - protein, 114 aa, synthetic construct
MAGLALQPGTALLCYSCKAQVSNEDCLQVENCTQLGEQCWTARIRAVGLLTVISKGCSLNCVDDSQDYYVGKKNITCCDTDLCNASGAHALQPAAAILALLPALGLLLWGPGQL